SEQUENCE LISTING (72 sequences)

SEQ ID NO: 1 - protein, 296 aa, Homo sapiens
MNAEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTSDPLEEDRFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 2 - DNA, 1070 bp, Homo sapiens
ggggtggcgcccggagctgcacggagagcgtgcccgtcagtgaccgaagaagagaccaagatgaatgcagagcccgagaggaagtttggcgtggtggtggttggtgttggccgagccggctccgtgcggatgagggacttgcggaatccacacccttcctcagcgttcctgaacctgattggcttcgtgtcgagaagggagctcgggagcattgatggagtccagcagatttctttggaggatgctctttccagccaagaggtggaggtcgcctatatctgcagtgagagctccagccatgaggactacatcaggcagttccttaatgctggcaagcacgtccttgtggaataccccatgacactgtcattggcggccgctcaggaactgtgggagctggctgagcagaaaggaaaagtcttgcacgaggagcatgttgaactcttgatggaggaattcgctttcctgaaaaaagaagtggtggggaaagacctgctgaaagggtcgctcctcttcacatctgacccgttggaagaagaccggtttggcttccctgcattcagcggcatctctcgactgacctggctggtctccctctttggggagctttctcttgtgtctgccactttggaagagcgaaaggaagatcagtatatgaaaatgacagtgtgtctggagacagagaagaaaagtccactgtcatggattgaagaaaaaggacctggtctaaaacgaaacagatatttaagcttccatttcaagtctgggtccttggagaatgtgccaaatgtaggagtgaataagaacatatttctgaaagatcaaaatatatttgtccagaaactcttgggccagttctctgagaaggaactggctgctgaaaagaaacgcatcctgcactgcctggggcttgcagaagaaatccagaaatattgctgttcaaggaagtaagaggaggaggtgatgtagcacttccaagatggcaccagcatttggttcttctcaagagttgaccattatctctattcttaaaattaaacatgttggggaaacaaaaaaaaaaaaaaaaa

SEQ ID NO: 3 - protein, 296 aa, Sus scrofa
MNAEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGSPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 4 - protein, 296 aa, Pan troglodytes
MNTEPERKFGVVVVGVGRAGSVRMRDLRNPHPSSAFLNLIGFVSRRELGSVDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 5 - protein, 296 aa, Macaca mulatta
MNTEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 6 - protein, 296 aa, Bos taurus
MNTEPERKFGVVVVGVGRAGSVRMRDLRNPHASSAFLNLIGFVSRRELGSIDEVPQISLEDALSSQEVEVAFICSESSSHEDYIRQFLNAGKHVLVEYPMTLSWVAAKDLWELAEQKGKVLHEEHVELLMEEFAFLKKEVVGKDLLKGSLLFTAAPLEEERFGFPAFSGISRLTWLVSLFGELSLVSATLEERKEDQYMKMTVCLETENKSPLTWIEEKAPGLKRNRRLSFHFRSGSLENMPNVGINKNIFLKDQNIFVQKLLGQFSEEELAAEKKRILHCLWLAGEIQKHCCSKQ

SEQ ID NO: 7 - protein, 7 aa, Artificial
  biliverdin reductase peptide
KXXXSXK

SEQ ID NO: 8 - protein, 7 aa, Artificial
  biliverdin reductase peptide
KYCCSRK

SEQ ID NO: 9 - protein, 7 aa, Artificial
  biliverdin reductase peptide
KSCCSRK

SEQ ID NO: 10 - protein, 7 aa, Artificial
  biliverdin reductase peptide
KTCCSRK

SEQ ID NO: 11 - protein, 7 aa, Artificial
  biliverdin reductase peptide
KYCCSKK

SEQ ID NO: 12 - protein, 7 aa, Artificial
  biliverdin reductase peptide
KSCCSKK

SEQ ID NO: 13 - protein, 7 aa, Artificial
  biliverdin reductase peptide
KTCCSKK

SEQ ID NO: 14 - protein, 7 aa, Artificial
  biliverdin reductase peptide
KYHCSRK

SEQ ID NO: 15 - protein, 7 aa, Artificial
  biliverdin reductase peptide
KSHCSRK

SEQ ID NO: 16 - protein, 7 aa, Artificial
  biliverdin reductase peptide
KTHCSRK

SEQ ID NO: 17 - protein, 7 aa, Artificial
  biliverdin reductase peptide
KYHCSKK

SEQ ID NO: 18 - protein, 7 aa, Artificial
  biliverdin reductase peptide
KSHCSKK

SEQ ID NO: 19 - protein, 7 aa, Artificial
  biliverdin reductase peptide
KTHCSKK

SEQ ID NO: 20 - protein, 7 aa, Artificial
  biliverdin reductase peptide
XXXXLXX

SEQ ID NO: 21 - protein, 7 aa, Artificial
  biliverdin reductase peptide
KKRILHC

SEQ ID NO: 22 - protein, 7 aa, Artificial
  biliverdin reductase peptide
RKRILCC

SEQ ID NO: 23 - protein, 7 aa, Artificial
  biliverdin reductase peptide
KRRILCC

SEQ ID NO: 24 - protein, 7 aa, Artificial
  biliverdin reductase peptide
KKRLLCC

SEQ ID NO: 25 - protein, 7 aa, Artificial
  biliverdin reductase peptide
RRRILCC

SEQ ID NO: 26 - protein, 7 aa, Artificial
  biliverdin reductase peptide
KRKILCC

SEQ ID NO: 27 - protein, 7 aa, Artificial
  biliverdin reductase peptide
RRRLLCC

SEQ ID NO: 28 - protein, 7 aa, Artificial
  biliverdin reductase peptide
KKKLLHC

SEQ ID NO: 29 - protein, 11 aa, Artificial
  biliverdin reductase peptide
XXXXLXXLXLA

SEQ ID NO: 30 - protein, 11 aa, Artificial
  biliverdin reductase peptide
KKRILCCLGLA

SEQ ID NO: 31 - protein, 11 aa, Artificial
  biliverdin reductase peptide
RKRILCCLGLA

SEQ ID NO: 32 - protein, 11 aa, Artificial
  biliverdin reductase peptide
KRRILCCLGLA

SEQ ID NO: 33 - protein, 11 aa, Artificial
  biliverdin reductase peptide
KKRLLCCLGLA

SEQ ID NO: 34 - protein, 11 aa, Artificial
  biliverdin reductase peptide
RRRILCCLGLA

SEQ ID NO: 35 - protein, 11 aa, Artificial
  biliverdin reductase peptide
KRKILCCLGLA

SEQ ID NO: 36 - protein, 11 aa, Artificial
  biliverdin reductase peptide
RRRLLCCLGLA

SEQ ID NO: 37 - protein, 11 aa, Artificial
  biliverdin reductase peptide
KKKLLCCLGLA

SEQ ID NO: 38 - protein, 8 aa, Artificial
  biliverdin reductase peptide
FXFPXFXX

SEQ ID NO: 39 - protein, 8 aa, Artificial
  biliverdin reductase peptide
FGFPAFSG

SEQ ID NO: 40 - protein, 8 aa, Artificial
  biliverdin reductase peptide
FGFPAFTG

SEQ ID NO: 41 - protein, 8 aa, Artificial
  biliverdin reductase peptide
FAFPGFSG

SEQ ID NO: 42 - protein, 8 aa, Artificial
  biliverdin reductase peptide
FAFPGFTG

SEQ ID NO: 43 - protein, 8 aa, Artificial
  biliverdin reductase peptide
FAFPAFTG

SEQ ID NO: 44 - protein, 8 aa, Artificial
  biliverdin reductase peptide
FGFPGFSG

SEQ ID NO: 45 - protein, 8 aa, Artificial
  biliverdin reductase peptide
FGFPGFTG

SEQ ID NO: 46 - protein, 12 aa, Artificial
  biliverdin reductase peptide
EEERFXFPXFXX

SEQ ID NO: 47 - protein, 7 aa, Artificial
  biliverdin reductase peptide
FXXFXFX

SEQ ID NO: 48 - protein, 7 aa, Artificial
  biliverdin reductase peptide
FAGFSFV

SEQ ID NO: 49 - protein, 7 aa, Artificial
  biliverdin reductase peptide
FAAFSFV

SEQ ID NO: 50 - protein, 7 aa, Artificial
  biliverdin reductase peptide
FGAFSFV

SEQ ID NO: 51 - protein, 7 aa, Artificial
  biliverdin reductase peptide
FAGFSFS

SEQ ID NO: 52 - protein, 7 aa, Artificial
  biliverdin reductase peptide
FAAFSFS

SEQ ID NO: 53 - protein, 7 aa, Artificial
  biliverdin reductase peptide
FGAFSFS

SEQ ID NO: 54 - protein, 7 aa, Artificial
  biliverdin reductase peptide
FAGFSFT

SEQ ID NO: 55 - protein, 7 aa, Artificial
  biliverdin reductase peptide
FAAFSFT

SEQ ID NO: 56 - protein, 7 aa, Artificial
  biliverdin reductase peptide
FGAFSFT

SEQ ID NO: 57 - RNA, 21 nt, Artificial
  biliverdin reductase siRNA
uccucagcguuccugaaccug

SEQ ID NO: 58 - RNA, 21 nt, Artificial
  biliverdin reductase siRNA
aggagucgcaaggacuuggac

SEQ ID NO: 59 - RNA, 27 nt, Artificial
  HO-2 siRNA
aggacuucuugaaaggcaacauuaaag

SEQ ID NO: 60 - RNA, 27 nt, Artificial
  HO-2 siRNA
uauccugaagaacuuuccguuguaauu

SEQ ID NO: 61 - DNA, 68 bp, Artificial
  biliverdin reductase siRNA oligonucleotide
gatcccctctgcagcattcctgaacctgttcaagagacaggttcaggaatgctgcagatttttggaaa

SEQ ID NO: 62 - DNA, 68 bp, Artificial
  biliverdin reductase siRNA oligonucleotide
agcttttccaaaaatctgcagcattcctgaacctgtctcttgaacaggttcaggaatgctgcagaggg

SEQ ID NO: 63 - DNA, 68 bp, Artificial
  HO-2 siRNA oligonucleotide
gatccccgaaaaccataccaaaatggcattcaagagatgccattttggtatggttttctttttggaaa

SEQ ID NO: 64 - DNA, 68 bp, Artificial
  HO-2 siRNA oligonucleotide
agcttttccaaaaagaaaaccataccaaaatggcatctcttgaatgccattttggtatggttttcggg

SEQ ID NO: 65 - DNA, 24 bp, Artificial
  GAPDH primer
tgatgctggtgctgagtatgtcgt

SEQ ID NO: 66 - DNA, 24 bp, Artificial
  GAPDH primer
ttgtcattgagagcaatgccagcc

SEQ ID NO: 67 - DNA, 24 bp, Artificial
  BVR primer
tgccgagccaaagaggaaatttgg

SEQ ID NO: 68 - DNA, 24 bp, Artificial
  BVR primer
agctgtgaagcgaagagacccttt

SEQ ID NO: 69 - DNA, 24 bp, Artificial
  HO-1 primer
ttaagctggtgatggcctccttgt

SEQ ID NO: 70 - DNA, 23 bp, Artificial
  HO-1 primer
catggccttctgcgcaatcttct

SEQ ID NO: 71 - DNA, 24 bp, Artificial
  HO-2 primer
aatggcagacctttctgagctcct

SEQ ID NO: 72 - DNA, 24 bp, Artificial
  HO-2 primer
gggcattgtccacatgctcaaaca

(19) United States
(12) Patent Application Publication
(10) Pub. No.: US 2010/0303790 A1
(43) Pub. Date: Dec. 2, 2010

(54) USE OF BILIVERDIN REDUCTASE (BVR) AND BVR PEPTIDE FRAGMENTS TO TREAT CORONARY DISORDERS

(75) Inventor: Mahin D. Maines, Rochester, NY (US)

Correspondence Address: NIXON PEABODY LLP - PATENT GROUP, 1100 CLINTON SQUARE, ROCHESTER, NY, 14604, US

(73) Assignee: UNIVERSITY OF ROCHESTER, Rochester, NY (US)

(21) Appl. No.: 12/775,218

(22) Filed: May 6, 2010

Related U.S. Application Data
(60) Provisional application No. 61/175,992, filed on May 6, 2009.

(51) Int. Cl. A61K 38/44; A61K 31/7088; A61P 9/00; C12N 9/02

(52) U.S. Cl. 424/94.4; 514/44 R; 514/44 A; 435/189

(57) ABSTRACT

The present invention relates to methods and compositions for treating coronary disorders and preventing cardiac cell death that involve the use of biliverdin reductase (“BVR”) or BVR peptides to stabilize the expression and activity of intracellular HO-2 in cardiac cells. The present invention also relates to methods and compositions for inducing cancer cell death in a patient having cancer that involve the use of inhibitory BVR peptides to destabilize intracellular HO-2 expression and activity, thereby augmenting cell death.

This application claims the priority benefit of U.S. Provisional Patent Application Ser. No. 61/175,992, filed May 6, 2009, which is hereby incorporated by reference in its entirety.

This invention was made with government support under ES012187 and ES004066 awarded by the National Institutes of Health, and grant number 0635312N from the American Heart Association National Center. The government has certain rights in this invention.

### FIELD OF THE INVENTION

The present invention relates to methods and compositions for modulating intracellular heme oxygenase-2 (“HO-2”) stabilization and activity that involve the use of biliverdin reductase (“BVR”) or BVR peptides. These methods and compositions are useful for the treatment of coronary disorders and cancer.

### BACKGROUND OF THE INVENTION

Heart failure represents the endpoint in most instances of cardiovascular pathology and remains a major cause of death in this country. More than 500,000 new cases of heart failure are reported each year and the 5 year survival rate is about 50%. Mammalian cardiomyocytes irreversibly withdraw from the cell cycle and undergo terminal differentiation soon after birth. Their limited regenerative capacity makes prevention of cardiomyocyte death key to the delay or prevention of heart failure. Hypertrophy, hypoxia, and free radical-mediated apoptosis of the cardiomyocytes are associated with activation of the β-adrenergic system.

β-adrenergic receptor activation by ligands, such as isoproterenol (ISO), mediates the activation of apoptosis in cardiac cells and also inotropic heart modification (Tomita et al., “Inducible cAMP Early Repressor (ICER) is A Negative-Feedback Regulator of Cardiac Hypertrophy and an Important Mediator of Cardiac Myocyte Apoptosis in Response to Beta-Adrenergic Receptor Stimulation,” Circ Res 93:12-22 (2003) and Zaugg et al., “Beta-Adrenergic Receptor Subtypes Differentially Affect Apoptosis in Adult Rat Ventricular Myocytes,” Circulation 102:344-350 (2000)). Cardiomyocyte apoptosis is preferentially dependent on activation of the β2-adrenergic receptor subtype in vitro, although both β1- and β2-adrenergic receptor subtypes coexist in cardiomyocytes (Bristow et al., “Beta 1- and Beta 2-Adrenergic-Receptor Subpopulations in Nonfailing and Failing Human Ventricular Myocardium: Coupling of Both Receptor Subtypes To Muscle Contraction and Selective Beta 1-Receptor Down-Regulation in Heart Failure,” Circ Res 59:297-309 (1986) and del Monte et al., “Coexistence of Functioning Beta 1- and Beta 2-Adrenoceptors in Single Myocytes From Human Ventricle,” Circulation 88:854-863 (1993)).

The present invention is directed to novel methods and compositions for preventing cardiac cell death and treating related coronary disorders.

### SUMMARY OF THE INVENTION

A first aspect of the present invention relates to a method of treating a coronary disorder that includes administering to a patient having a coronary disorder an effective amount of an agent that induces the stabilization of heme oxygenase-2 (HO-2) in cardiac cells, where said administering is effective to increase the level of HO-2 in cardiac cells and treat the coronary disorder.

Another aspect of the present invention relates to a method of preventing cardiac cell death that includes contacting a cardiac cell with an agent that induces the stabilization of HO-2 in cardiac cells, where said contacting is effective to increase the level of HO-2 in the cardiac cell and prevent cardiac cell death.

Another aspect of the present invention relates to a method of inducing cancer cell death in a patient. This method involves administering to a patient having cancer an effective amount of an agent that induces the destabilization of HO-2, where said administering is effective to decrease the level of HO-2 in cancer cells of the patient and thereby induce cancer cell death.

Another aspect of the present invention relates to an isolated peptide having an amino acid sequence of KX[C/H][C/H]SXK (SEQ ID NO:7), where X at position 2 is a tyrosine (Y), threonine (T), or serine (S), and X as position 6 is a positively charged amino acid, preferably arginine (R), or lysine (K).

Another aspect of the present invention relates to an isolated peptide comprising the amino acid sequence XXX[I/L]LXX (SEQ ID NO:20), wherein X at the positions 1, 2, and 3 is a positively charged amino acid residue, preferably K or R, X at position 6 is any amino acid, preferably cysteine (C) or histidine (H), and X at position 7 is any amino acid, preferably C.

The studies described herein identify a role of BVR in regulating HO-2 stabilization and expression within a cell. As demonstrated herein, the administration of small BVR peptides that induce HO-2 stabilization facilitate HO-2 mediated cardiac cell protection by inhibiting the progression of cardiac cell death. These peptides provide a novel therapeutic approach for the treatment of coronary diseases. Alternatively, the administration of small inhibitory BVR peptides prevent HO-2 stabilization and augment cell death. Using cell-specific targeting strategies, these inhibitory peptides provide a means for inducing cancer cell specific death in a patient having cancer to reduce and/or prevent tumor growth and progression.

### BRIEF DESCRIPTION OF THE DRAWINGS

FIG. 1 is a Clustal alignment showing the homology between several mammalian BVR amino acid sequences including human (SEQ ID NO:1), pig (SEQ ID NO:3), chimp (SEQ ID NO:4), rhesus monkey (SEQ ID NO:5), and cattle (SEQ ID NO:6)

FIGS. 2A-2D show isoproterenol mediated increase in BVR and HO-2 expression in rat neonatal cardiomyocytes. The effect of isoproterenol on HO-2 and BVR protein levels is shown in the immunoblot of FIG. 2A. Cells were treated with 10 μM isoproterenol (ISO) for the times indicated, lysed, and proteins were resolved by SDS-PAGE. The western blot was probed sequentially with antibodies to rat BVR, rat HO-2 and β-actin, as a control. HO-2 and BVR mRNA expression (RT-PCR) in isoproterenol-stimulated rat neonatal cardiomyocytes is shown in FIG. 2B. RNA was isolated at the indicated times following isoproterenol exposure, and reverse transcribed. The rat BVR and HO-2 sequences were amplified by PCR, and products were resolved by agarose gel electrophoresis. GAPDH was included as a control. FIG. 2C is an agarose gel showing isoproterenol mediated transient induction of HO-1 RNA. Cardiomyocytes were treated with 10 μM ISO for the times indicated; RNA was isolated and the levels of HO-1 and GAPDH mRNAs determined as in described above. As depicted in the western blot of FIG. 2D, induction of HO-2 synthesis by ISO requires the induction of BVR. Cardiomyocytes were treated with si-rBVR retrovirus, and 12 hours later, with 10 μM ISO for an additional 24 hours. si-rBVR was replenished every 12 hours and proteins were resolved by gel electrophoresis and analyzed by western blot.

FIGS. 3A-3B show the increased expression of BVR and HO-2 after continuous cardiac infusion of isoproterenol in vivo. The levels of HO-2 and BVR protein in heart tissue from four individual rats after four days of continuous infusion with 400 μg isoproterenol/kg/h, or from four sham-operated rats were determined by western blot (FIG. 3A). The level of β-actin protein was used as a control. HO-2 and BVR mRNA expression in isoproterenol infused rat heart in vivo, determined by RT-PCR and agarose gel electrophoresis, is shown in FIG. 3B. Animals were infused with ISO as described above. The level of GAPDH mRNA is included as a control.

FIG. 4 is a western blot showing the involvement of PKA in mediating the increase in BVR and HO-2 protein levels. Cardiomyocytes were infected with an adenovirus expressing a PKA inhibitor (PKA-I) or a control gene (lacZ), then treated with 10 μM isoproterenol for the indicated times. BVR and HO-2 were detected in cell lysates by a western blot, using β-actin as a control.

FIGS. 5A-5D demonstrate that ablation of BVR protein expression prevents isoproterenol-induced HO-2 protein expression. Cardiomyocytes were infected with retrovirus expressing si-rBVR, then treated with 10 μM isoproterenol 6 h later. Samples were collected at 24, 48, 72 and 96 hours. Cell lysates were analysed by gel electrophoresis and the resulting immunoblots were probed with antibodies against rat BVR and HO-2 as shown in FIG. 5A. β-actin served as a control. In FIG. 5B, cardiomyocytes were infected with a retrovirus expressing si-rHO-2 prior to treatment with isoproterenol. Expression of BVR and HO-2 was measured at 24, 48, 72, and 96 hours. As illustrated in FIG. 5C, BVR expression regulates the stability of the HO-2 protein. In the first experiment (top three immunoblots), HEK293A cells were co-transfected with pcDNA-hBVR and pcDNA-hHO-2, then stimulated with isoproterenol for 24 h followed by treatment with the protein synthesis inhibitor cycloheximide (10 μg/ml) for the indicated times. The expression of hHO-2 and hBVR in HEK293A cells was measured by western blot. In the second series of experiments (bottom three immunoblots), HEK293A cells were transfected with pcDNA-hHO-2 and treated with retrovirus expressing si-hBVR. These data were replicated in at least two independent experiments. In FIG. 5D, cardiomyocytes were treated with 10 μM isoproterenol, infected with si-rBVR virus, or treated with the proteasome inhibitor MG132 (50 μg/ml). HO-2 and BVR protein expression was detected by western blot. β-actin served as a control.

FIGS. 6A-6D show that inhibition of BVR and HO-2 protein expression during isoproterenol stimulation increases cardiomyocyte apoptosis. In FIG. 6A, cardiomyocytes were treated with siRNA against rat BVR, HO-2, or both, after which isoproterenol was added at a final concentration of 10 μM, and the cells were incubated for 24 hours. A randomized form of si-hBVR (sc-BVR) was added to some cells as a control. Expression of BVR, HO-2, and cleaved caspase-3 was determined by western blot. β-actin served as a loading control. Quantitative analysis of the number of apoptotic cells detected, in cultures treated with isoproterenol and si-rBVR, si-rHO-2, or both si-rBVR and si-rHO-2 is shown in FIG. 6B. sc-hBVR served as control. The number of TUNEL-positive cells that also stained with EA-53 anti-α-actinin antibody was measured as a fraction of all cardiomyocytes. FIG. 6C is a series of fluorescent photomicrographs showing the detection of apoptosis in cardiomyocytes. Cardiomyocytes were characterized by their staining with antibody against α-actinin. Apoptosis was detected by TUNEL staining, and cell nuclei were visualized with DAPI. FIG. 6D is an agarose gel showing the electrophoresis of total genomic DNA isolated from cardiomyocytes that had been treated with ISO and si-BVR, si-HO-2, or both. Fragmentation of nuclear DNA is detectable in ISO-treated cardiomyocytes that were also treated with siBVR, siHO-2, or both. The 1 kb-plus ladder was used as DNA marker.

FIGS. 7A-7D show peptide-mediated inhibition of BVR activation in perfused heart increases cardiomyocyte apoptosis, whereas BVR activation is protective. In FIG. 7A, three groups of four rats each were injected subcutaneously with ISO (0.01 mg/kg), twice, with a 6 hour interval between injections prior to perfusion of the isolated hearts. As indicated in the Examples infra, isolated hearts were perfused with the peptides KKRILHC²⁸¹ (SEQ ID NO:21) (25 μM) or KYCCSRK²⁹⁶ (SEQ ID NO:8) in the presence or absence of 0.1 μM isoproterenol, for 3 hours. The levels of BVR, HO-2, and cleaved caspase-3 were determined by western blot, using β-actin as a control for equal loading. FIG. 7B is a graph showing BVR activity measured by the rate of conversion of biliverdin to bilirubin at pH 6.7, using NADH as cofactor. The rate of conversion of biliverdin to bilirubin was determined from the increase in absorbance at 450 nm at 25° C. Specific activity is expressed as nanomoles of bilirubin/min/mg of protein. FIG. 7C is a graph tracking the detection of apoptosis in cardiomyocytes from the perfused heart. Tissue from the perfused hearts was analysed using the TUNEL assay, as described in FIG. 6B. FIG. 7D is an agarose gel showing the resolution of genomic DNA isolated from perfused heart; fragmentation is detectable only in hearts perfused with ISO and the peptide KKRILHC²⁸¹ (SEQ ID NO:21). The 1 kb-plus ladder DNA was used as a marker.

FIGS. 8A-8C demonstrate that inhibition of BVR activation exacerbates ISO-mediated decline in the contractile function of the heart and BVR activation protects against loss of heart function. Heart rate (FIG. 8A, top graph) and left ventricular systolic pressure (FIG. 8A, bottom graph) were measured simultaneously during perfusion of the heart with ISO alone (▪) or in combination with peptide KKRILHC²⁸¹ (▴, SEQ ID NO:8) or KYCCSRK²⁹⁶ (◯, SEQ ID NO:21) (n=4). Developed pressure, calculated as the difference between the end systolic pressure and end diastolic pressure for the left ventricle, for the same animals as described above is shown in FIG. 8B. The first derivatives of left ventricular systolic pressure, dp/dt and −dp/dt are shown in FIG. 8C.

### DETAILED DESCRIPTION OF THE INVENTION

The present invention relates generally to methods of modulating (i.e., increasing or decreasing) intracellular heme oxygenase-2 (HO-2) stabilization, expression, and/or activity to regulate cellular protection or augment cell death. Enhancing HO-2 stabilization and expression within a cell facilitates cellular protection and prevents cell death. Enhancing HO-2 destabilization and/or reducing HO-2 expression can induce or hasten cell death that is induced by an apoptotic agent or stimuli.

As described below, cells of interest where HO-2 stabilization is desired include cells that are otherwise healthy cells exposed to hypoxia during ischemic events, including, without limitation, cardiac cells (e.g., cardiomyocytes) and vascular cells (e.g., endothelial or vascular smooth muscles cells) of the heart. Cells of interest where HO-2 destabilization is desired include cancer cells, such as, ovarian cancer cells, esophageal cancer cells, colorectal cancer cells, prostate cancer cells, renal cancer cells, pancreatic cancer cells, breast cancer cells, skin cancer cells, oral cavity cancer cells, lung cancer cells, gastrointestinal cancer cells, liver cancer cells, head and neck cancer cells, and brain cancer cells.

HO-1 and HO-2, also known as the HSP32 family of proteins, and biliverdin reductase (“BVR”) are active participants in the catalytic conversion of the heme molecule to bile pigments. A number of studies have asserted that activation of the stress-inducible cognate of HSP32 family of proteins, HO-1, is cardio-protective. Unlike HO-1, HO-2 is generally considered to be the constitutively expressed member of the family, with cell type and tissue-dependent levels of expression (Ewing et al., “In Situ Hybridization and Immunohistochemical Localization of Heme Oxygenase-2 mRNA and Protein in Normal Rat Brain: Differential Distribution of Isozyme 1 and 2,” Mol Cell Neurosci 3:559-570 (1992), which is hereby incorporated by reference in its entirety).

BVR has been recently identified as a multi-functional enzyme, with one key function involving the reduction of biliverdin, the product of heme oxidation, to bilirubin. The other functions of human BVR that have been identified include its role as a dual-specific kinase, transcription factor, and molecular scaffold and cellular transporter of kinases and regulatory factors (Florczyk et al., “Biliverdin Reductase: New Features of an Old Enzyme and Its Potential Therapeutic Significance,” Pharmacol Rep 60:38-48 (2008) and Kapitulnik et al., “Pleiotropic Functions of Biliverdin Reductase: Cellular Signaling and Generation of Cytoprotective and Cytotoxic Bilirubin,” Trends Pharmacol Sci 30:129-137 (2009), which are hereby incorporated by reference in their entirety).

HO-2 is prominently expressed in the cardiovascular system, including in the endothelial cells of the carotid artery (Ewing et al., “Induction of Heart Heme Oxygenase-1 (HSP32) by Hyperthermia: Possible Role in Stress-Mediated Elevation of Cyclic 3′,5′-Guanosine Monophosphate,” J Pharmacol Exp Ther 271:408-414 (1994), which is hereby incorporated by reference in its entirety), in glomus cells of the carotid body (Prabhakar et al., “Carbon Monoxide: A Role in Carotid Body Chemoreception,” Proc Natl Acad Sci USA 92:1994-1997 (1995), which is hereby incorporated by reference in its entirety), in the brain and the nervous system, including the hippocampus (Ewing et al., “In Situ Hybridization and Immunohistochemical Localization of Heme Oxygenase-2 mRNA and Protein in Normal Rat Brain: Differential Distribution of Isozyme 1 and 2,” Mol Cell Neurosci 3:559-570 (1992) and Ewing et al., “Rapid Induction of Heme Oxygenase 1 mRNA and Protein by Hyperthermia in Rat Brain: Heme Oxygenase 2 Is Not A Heat Shock Protein,” Proc Natl Acad Sci USA 88:5364-5368 (1991), which are hereby incorporated by reference in their entirety), in the spinal cord (Panahian et al., “Site of Injury-Directed Induction of Heme Oxygenase-1 and -2 in Experimental Spinal Cord Injury: Differential Functions in Neuronal Defense Mechanisms?” J Neurochem 76:539-554 (2001), which is hereby incorporated by reference in its entirety), and in the interstitial cell network of the small intestine (Miller et al., “Heme Oxygenase 2 Is Present in Interstitial Cell Networks of the Mouse Small Intestine,” Gastroenterology 114:239-244 (1998), which is hereby incorporated by reference in its entirety).

Like BVR, HO-2 also plays a pivotal role in cellular homeostasis. Both isozymes of HO(HO-1 and HO-2) catalyze oxidative cleavage of heme molecules (Fe-protoporphyrin-IX) to carbon monoxide (CO) and biliverdin, two biologically active molecules. Carbon monoxide, similar to nitric oxide (NO), is a signaling molecule in the brain and the cardiovascular system (Verma et al., “Carbon Monoxide: A Putative Neural Messenger,” Science 259:381-384 (1993); Ewing et al., “Induction of Heart Heme Oxygenase-1 (HSP32) by Hyperthermia: Possible Role in Stress-Mediated Elevation of Cyclic 3′,5′-Guanosine Monophosphate,” J Pharmacol Exp Ther 271:408-414 (1994); Prabhakar N., “NO and CO as Second Messengers in Oxygen Sensing in the Carotid Body,” Respir Physiol 115:161-168 (1999); and Prabhakar et al., “Carbon Monoxide: A Role in Carotid Body Chemoreception,” Proc Natl Acad Sci USA 92:1994-1997 (1995), which are hereby incorporated by reference in their entirety), while biliverdin is precursor to the formation of potent intracellular antioxidant bilirubin (Ryter et al., “Carbon Monoxide and Bilirubin: Potential Therapies for Pulmonary/Vascular Injury and Disease,” Am J Respir Cell Mol Biol 36:175-182 (2007) and Maghzal et al., “Limited Role for the Bilirubin-Biliverdin Redox Amplification Cycle in the Cellular Antioxidant Protection by Biliverdin Reductase,” J Biol Chem 284:29251-29259 (2009), which are hereby incorporated by reference in their entirety).

In addition to its activity in the heme degradation pathway, HO-2 performs functions that are exclusive to this form of heme oxygenase; this in turn, reflects the primary structural features of the protein. HO-2 is among a select group of proteins that possess the so-called heme regulating motifs (HRM) which have the core sequence of Cys-Pro. The dipeptide is flanked upstream with positively charged residues and downstream by hydrophobic amino acids; two copies of HRM motifs are present in HO-2 (Rotenberg et al., “Isolation, Characterization, and Expression in Escherichia coli of a cDNA Encoding Rat Heme Oxygenase-2,” J Biol Chem 265:7501-7506 (1990), which is hereby incorporated by reference in its entirety). The amino acid composition provides suitable configuration for heme binding (Rublevskaya et al., “Interaction of Fe-Protoporphyrin IX and Heme Analogues with Purified Recombinant Heme Oxygenase-2, the Constitutive Isozyme of the Brain and Testes,” J Biol Chem 269:26390-26395 (1994) and McCoubrey et al., “Heme Oxygenase-2 is a Hemoprotein and Binds Heme Through Heme Regulatory Motifs that are not Involved in Heme Catalysis,” J Biol Chem 272:12568-12574 (1997), which are hereby incorporated by reference in their entirety). The two HRMs, which are separated by 17 amino acids, can also form a disulfide bond and, as such, provide a redox sensitive molecular target (Yi et al., “Heme Regulatory Motifs in Heme Oxygenase-2 Form a Thiol/Disulfide Redox Switch That Responds to the Cellular Redox State,” J Biol Chem 284:20556-20561 (2009), which is hereby incorporated by reference in its entirety) as well as a site of interaction with nitric oxide. In the brain, HO-2 is associated with the calcium-dependent potassium channels, and potentially serves as the oxygen sensor (Maines M., “The Heme Oxygenase System: A Regulator of Second Messenger Gases,” Annu Rev Pharmacol Toxicol 37:517-554 (1997); Williams et al., “Hemoxygenase-2 Is an Oxygen Sensor for a Calcium-Sensitive Potassium Channel,” Science 306:2093-2097 (2004); and Kemp P., “Hemeoxygenase-2 as an O₂Sensor in K⁺ Channel-Dependent Chemotransduction,”Biochem Biophys Res Commun 338:648-652 (2005), which are hereby incorporated by reference in their entirety). HO-2 is a phosphoprotein and a substrate for BVR (Lerner-Marmarosh et al., “Human Biliverdin Reductase: A Member of the Insulin Receptor Substrate Family with Serine/Threonine/Tyrosine Kinase Activity,” Proc Natl Acad Sci USA 102:7109-7114 (2005), which is hereby incorporated by reference in its entirety).

To manipulate the level of HO-2 present in cells of interest, the concentration of activated HO-2 can be manipulated directly by HO-2 gene therapy. The nucleic acid sequence encoding human and other mammalian HO-2 proteins are known in the art (see, e.g., GenBank Accession Nos. NM_{—}001127204 (human), XM_{—}001168788 (chimpanzee), NM_{—}001136066 (mouse), and NM_{—}024387 (rat), which are hereby incorporated by reference in their entirety), and inhibitory nucleic acid molecules (e.g., antisense molecules, short interfering RNA molecules (siRNA), short hairpin RNA molecules (shRNA), and microRNA molecules (miRNA)) specific for down-regulating HO-2 are also known. Methods of producing recombinant nucleic acid molecules and gene therapy vectors are disclosed infra.

Alternatively, the concentration of activated HO-2 can be manipulated by upregulating the concentration of activated BVR, mimicking BVR activity, or inhibiting BVR activity in those target cells. Increasing the concentration of activated cellular BVR or mimicking BVR activity increases intracellular HO-2 stabilization and expression. This can be achieved by, for example, introducing BVR or BVR-derived peptides into the cells of interest either directly or by recombinant expression of nucleic acid molecule encoding BVR or BVR-derived peptides (i.e., via gene therapy). The concentration of activated cellular BVR can also be increased by introducing an agent, e.g., a small molecule or upstream signaling molecule, that increases BVR transcription and expression or activity in a cell. In contrast, decreasing the concentration of activated cellular BVR or inhibiting BVR activity decreases intracellular HO-2 stabilization and expression. This can be achieved by, for example, introducing an inhibitory BVR peptide into the cell of interest either directly or by recombinant expression of nucleic acid molecule encoding the inhibitory BVR peptide. Alternatively, inhibition of BVR activity can be achieved by introducing an agent, e.g., a small molecule or inhibitory nucleic acid molecule (e.g., siRNA, antisense RNA, shRNA, or miRNA), into the target cells of interest.

As used herein, the terms “biliverdin reductase” and “BVR” refer to any mammalian BVR, but preferably human BVR (“hBVR”). One form of hBVR has an amino acid sequence corresponding to SEQ ID NO: 1 as follows:

```
Met Asn Ala Glu Pro Glu Arg Lys Phe Gly Val Val Val Val Gly Val
    1 5 10 15
Gly Arg Ala Gly Ser Val Arg Met Arg Asp Leu Arg Asn Pro His Pro
                          20 25 30
Ser Ser Ala Phe Leu Asn Leu Ile Gly Phe Val Ser Arg Arg Glu Leu
                  35 40 45
Gly Ser Ile Asp Gly Val Gln Gln Ile Ser Leu Glu Asp Ala Leu Ser
          50 55 60
Ser Gln Glu Val Glu Val Ala Tyr Ile Cys Ser Glu Ser Ser Ser His
  65 70 75 80
Glu Asp Tyr Ile Arg Gln Phe Leu Asn Ala Gly Lys His Val Leu Val
                                  85 90 95
Glu Tyr Pro Met Thr Leu Ser Leu Ala Ala Ala Gln Glu Leu Trp Glu
                        100 105 110
Leu Ala Glu Gln Lys Gly Lys Val Leu His Glu Glu His Val Glu Leu
                115 120 125
Leu Met Glu Glu Phe Ala Phe Leu Lys Lys Glu Val Val Gly Lys Asp
        130 135 140
Leu Leu Lys Gly Ser Leu Leu Phe Thr Ser Asp Pro Leu Glu Glu Asp
145 150 155 160
Arg Phe Gly Phe Pro Ala Phe Ser Gly Ile Ser Arg Leu Thr Trp Leu
                                165 170 175
Val Ser Leu Phe Gly Glu Leu Ser Leu Val Ser Ala Thr Leu Glu Glu
                        180 185 190
Arg Lys Glu Asp Gln Tyr Met Lys Met Thr Val Cys Leu Glu Thr Glu
                195 200 205
Lys Lys Ser Pro Leu Ser Trp Ile Glu Glu Lys Gly Pro Gly Leu Lys
        210 215 220
Arg Asn Arg Tyr Leu Ser Phe His Phe Lys Ser Gly Ser Leu Glu Asn
225 230 235 240
Val Pro Asn Val Gly Val Asn Lys Asn Ile Phe Leu Lys Asp Gln Asn
                                245 250 255
Ile Phe Val Gln Lys Leu Leu Gly Gln Phe Ser Glu Lys Glu Leu Ala
                        260 265 270
Ala Glu Lys Lys Arg Ile Leu His Cys Leu Gly Leu Ala Glu Glu Ile
                275 280 285
Gln Lys Tyr Cys Cys Ser Arg Lys
        290 295
```

Heterologous expression and isolation of hBVR is described in Maines et al., Human Biliverdin IXalpha Reductase is a Zinc-Metalloprotein. Characterization of Purified and Escherichia coli Expressed Enzymes,” Eur. J. Biochem. 235(1-2):372-381 (1996) and Maines et al., “Purification and Characterization of Human Biliverdin Reductase,” Arch. Biochem. Biophys. 300(1):320-326 (1993), which are hereby incorporated by reference in their entirety. A nucleic acid molecule encoding this form of hBVR has a nucleotide sequence corresponding to SEQ ID NO: 2 as follows:

| | |
|---|---|
| ggggtggcgc ccggagctgc acggagagcg tgcccgtcag tgaccgaaga agagaccaag | 60 |
| atgaatgcag agcccgagag gaagtttggc gtggtggtgg ttggtgttgg ccgagccggc | 120 |
| tccgtgcgga tgagggactt gcggaatcca cacccttcct cagcgttcct gaacctgatt | 180 |
| ggcttcgtgt cgagaaggga gctcgggagc attgatggag tccagcagat ttctttggag | 240 |
| gatgctcttt ccagccaaga ggtggaggtc gcctatatct gcagtgagag ctccagccat | 300 |
| gaggactaca tcaggcagtt ccttaatgct ggcaagcacg tccttgtgga ataccccatg | 360 |
| acactgtcat tggcggccgc tcaggaactg tgggagctgg ctgagcagaa aggaaaagtc | 420 |
| ttgcacgagg agcatgttga actcttgatg gaggaattcg ctttcctgaa aaaagaagtg | 480 |
| gtggggaaag acctgctgaa agggtcgctc ctcttcacat ctgacccgtt ggaagaagac | 540 |
| cggtttggct tccctgcatt cagcggcatc tctcgactga cctggctggt ctccctcttt | 600 |
| ggggagcttt ctcttgtgtc tgccactttg gaagagcgaa aggaagatca gtatatgaaa | 660 |
| atgacagtgt gtctggagac agagaagaaa agtccactgt catggattga agaaaaagga | 720 |
| cctggtctaa aacgaaacag atatttaagc ttccatttca agtctgggtc cttggagaat | 780 |
| gtgccaaatg taggagtgaa taagaacata tttctgaaag atcaaaatat atttgtccag | 840 |
| aaactcttgg gccagttctc tgagaaggaa ctggctgctg aaaagaaacg catcctgcac | 900 |
| tgcctggggc ttgcagaaga aatccagaaa tattgctgtt caaggaagta agaggaggag | 960 |
| gtgatgtagc acttccaaga tggcaccagc atttggttct tctcaagagt tgaccattat | 1020 |
| ctctattctt aaaattaaac atgttgggga aacaaaaaaa aaaaaaaaaa | 1070 |

The open reading frame which encodes hBVR of SEQ ID NO: 1 extends from nucleotide position 1 to nucleotide position 888 of SEQ ID NO:2.

Another form of hBVR has been reported by Komuro et al., NCBI Accession No. G02066, direct submission to the EMBL Data Library (1998) (“Komuro”), which is hereby incorporated by reference in its entirety. Differences between the hBVR of SEQ ID NO: 1 and the hBVR of Komuro et al. are at amino acid residues 3, 154, 155, and 160. Specifically, residue 3 can be either alanine or threonine, residue 154 can be either alanine or serine, residue 155 can be either aspartic acid or glycine, and residue 160 can be either aspartic acid or glutamic acid.

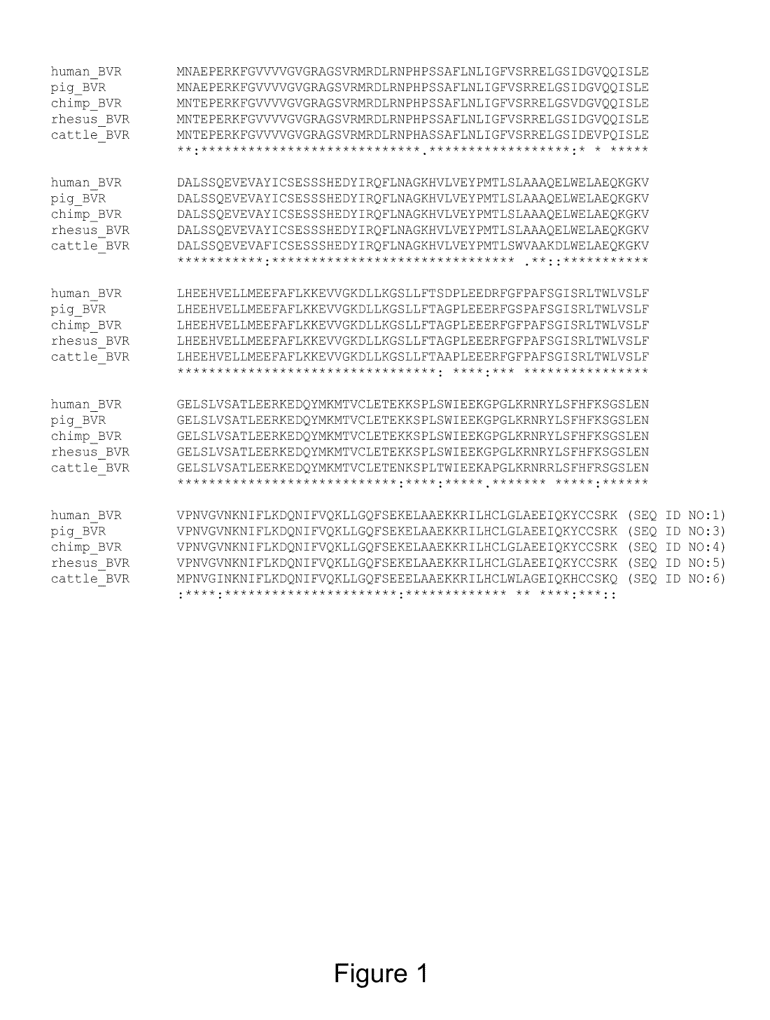

In addition, BVR from other mammals, such as rat, mouse, pig, and chimp that have been recombinantly expressed and isolated (see e.g., Fakhrai et al., “Expression and Characterization of a cDNA for Rat Kidney Biliverdin Reductase. Evidence Suggesting the Liver and Kidney Enzymes are the Same Transcript Product,” J. Biol. Chem. 267(6):4023-4029 (1992), which is hereby incorporated by reference in its entirety) can be employed in the methods of the present invention. The rBVR of shares about 82% amino acid identity to the hBVR of SEQ ID NO: 1, with variations in amino acid residues being highly conserved. The mouse BVR sequence (Genbank Accession NP_{—}080954, which is hereby incorporated by reference in its entirety) is about 81 percent identical to the human BVR sequence of SEQ ID NO: 1, with variations in amino acid residues being highly conserved. The pig, chimp (Genbank Accession XP_{—}001136150, which is hereby incorporated by reference in its entirety), and rhesus monkey (Genbank Accession XP_{—}001095668, which is hereby incorporated by reference in its entirety) BVR sequences are each about 98 percent identical to the human BVR sequence of SEQ ID NO: 1, with variations in amino acid residues being highly conserved. The cattle BVR sequences is about 95 percent identical to the human BVR sequence of SEQ ID NO: 1, with variations in amino acid residues being highly conserved. The relatedness of several of the mammalian BVR proteins is illustrated in the Clustal alignment presented FIG. 1.

It is to be understood that the present invention contemplates the use of any mammalian or non-mammalian BVR sequence in the formation of the recombinant genes, expression systems, and peptides of the present invention. Homologous BVR peptides from mammals and non-mammals other than those described above are preferably characterized by an amino acid identity of at least about 60 percent, more preferably at least about 70 percent or 80 percent, most preferably at least about 85 percent or 90 percent or 95 percent as compared to human BVR of SEQ ID NO: 1. Other mammalian and non-mammalian cDNA molecules can be identified based upon their alignment with the BVR cDNA of SEQ ID NO: 2, where such alignment preferably is at least about 60 percent identical more preferably at least about 70 percent, 75 percent, 80 percent, 85 percent, 90 percent, or 95 percent identical. Alternatively, other mammalian BVR encoding cDNA molecules can be identified by the ability of mammalian cDNA sequences to hybridize to the complement of SEQ ID NO: 2 under stringent hybridization and wash conditions. Exemplary stringent hybridization and wash conditions include, without limitation, hybridization at 50° C. or higher (i.e., 55° C., 60° C., or 65° C.) in a hybridization medium that includes 0.9× (or higher, such as 2× or 5×) sodium citrate (“SSC”) buffer, followed by one or more washes at increasing stringency using 0.2×SSC buffer at temperatures from 42° C. up to the temperature of the hybridization step. Higher stringency can readily be attained by increasing the temperature for either hybridization or washing conditions or decreasing the sodium concentration of the hybridization or wash medium. Nonspecific binding may also be controlled using any one of a number of known techniques such as, for example, blocking the membrane with protein-containing solutions, addition of heterologous RNA, DNA, and SDS to the hybridization buffer, and treatment with RNase. Wash conditions are typically performed at or below stringency.

Various BVR peptides can be employed to modulate intracellular HO-2 concentration and/or stability. A BVR peptide useful for stabilizing HO-2 expression and activity has an amino acid sequence of KX[C/H][C/H]SXK (SEQ ID NO:7), where X at position 2 is a tyrosine (Y), threonine (T), or serine (S), and X as position 6 is a positively charged amino acid, preferably arginine (R), or lysine (K). This peptide is preferably myristolated or acetylated. Exemplary peptides include, without limitation, KYCCSRK (SEQ ID NO:8), KSCCSRK (SEQ ID NO:9), KTCCSRK (SEQ ID NO:10), KYCCSKK (SEQ ID NO:11), KSCCSKK (SEQ ID NO:12), KTCCSKK (SEQ ID NO:13), KYHCSRK (SEQ ID NO:14), KSHCSRK (SEQ ID NO:15), KTHCSRK (SEQ ID NO:16), KYHCSKK (SEQ ID NO:17), KSHCSKK (SEQ ID NO:18), KTHCSKK (SEQ ID NO:19).

BVR peptides that destabilize HO-2 expression and activity are also encompassed by the present invention. In one embodiment, the BVR peptide that destabilizes HO-2 expression and activity has an amino acid sequence of XXX[I/L]LXX (SEQ ID NO:20), wherein X at the positions 1, 2, and 3 is a positively charged amino acid residue, preferably K or R, X at position 6 is any amino acid, preferably cysteine (C) or histidine (H), and the X at position 7 is any amino acid, but preferably C. Exemplary peptides include, without limitation, KKRILHC (SEQ ID NO:21), RKRILCC (SEQ ID NO:22), KRRILCC (SEQ ID NO:23), KKRLLCC (SEQ ID NO:24), RRRILCC (SEQ ID NO:25), KRKILCC (SEQ ID NO:26), RRRLLCC (SEQ ID NO:27), and KKKLLHC (SEQ ID NO:28). These peptides may be acetylated or myristoylated. In another embodiment, the BVR peptide that destabilizes HO-2 expression and activity has an amino acid sequence of XXX[I/L]LXXLXLA (SEQ ID NO:29), where X at positions 1, 2, and 3 is a positively charged amino acid residue, preferably K or R, X at position 6 is any amino acid residue, preferably C or H, X at position 7 is any amino acid residue, preferably C, and X at position 9 is any amino acid residue, preferably glycine. Exemplary BVR peptides encompassed by this consensus sequence include, without limitation, KKRILCCLGLA (SEQ ID NO:30), RKRILCCLGLA (SEQ ID NO:31), KRRILCCLGLA (SEQ ID NO:32), KKRLLCCLGLA (SEQ ID NO:33), RRRILCCLGLA (SEQ ID NO:34), KRKILCCLGLA (SEQ ID NO:35), RRRLLCCLGLA (SEQ ID NO:36), KKKLLCCLGLA (SEQ ID NO:37). Other peptides encompassed by the above consensus amino acid sequences are also within the scope of the present invention.

In another embodiment of the present invention, the BVR peptide that destabilizes HO-2 expression and activity has a consensus amino acid sequence of FXFPXF[S/T]X (SEQ ID NO:38), where X at amino acid positions 2 and 5 is any amino acid, preferably G or alanine (A), and X at position 8 is any amino acid, preferable G or valine (V). Exemplary BVR peptides encompassed by this consensus sequence include, without limitation, FGFPAFSG (SEQ ID NO:39), FGFPAFTG (SEQ ID NO:40), FAFPGFSG (SEQ ID NO:41), FAFPGFTG (SEQ ID NO:42), FAFPAFTG (SEQ ID NO:43), FGFPGFSG (SEQ ID NO:44), and FGFPGFTG (SEQ ID NO:45). Other peptides encompassed by the above consensus amino acid sequence are also within the scope of the present invention. The above peptides may further comprise one to four upstream charged amino acid residues, preferably glutamic acid or arginine residues, e.g., EEERFXFPXF[S/T]X (SEQ ID NO:46).

In another embodiment, the BVR peptide that destabilizes HO-2 expression and activity has a consensus amino acid sequence of FXXFXFX (SEQ ID NO:47), where X at amino acid positions 2 and 3 is any amino acid residue, preferably G or A, X at amino acid position 5 is any amino acid, preferably S or T, and X at position 7 is S, T, or V. Exemplary BVR peptide encompassed by this consensus sequence include, without limitation, FAGFSFV (SEQ ID NO:48), FAAFSFV (SEQ ID NO:49), FGAFSFV (SEQ ID NO:50), FAGFSFS (SEQ ID NO:51), FAAFSFS (SEQ ID NO:52), FGAFSFS (SEQ ID NO:53), FAGFSFT (SEQ ID NO:54), FAAFSFT (SEQ ID NO:55), FGAFSFT (SEQ ID NO:56). Other peptides encompassed by the above consensus amino acid sequence are also within the scope of the present invention.

In another embodiment of the present invention, the destabilization of intracellular HO-2 can also be achieved via the use of inhibitory BVR nucleic acid molecules or inhibitory HO-2 nucleic acid molecules, including, without limitation, antisense molecules, siRNA molecules, shRNA molecules, and miRNA molecules.

Antisense nucleic acid molecule capable of hybridizing with an RNA transcript coding for BVR are expressed from a transgene which is prepared by ligation of a DNA molecule, coding for BVR, or a fragment or variant thereof, into an expression vector in reverse orientation with respect to its promoter and 3′ regulatory sequences. Upon transcription of the DNA molecule, the resulting RNA molecule will be complementary to the mRNA transcript coding for the actual protein or polypeptide product. Ligation of DNA molecules in reverse orientation can be performed according to known techniques which are standard in the art. As discussed infra, recombinant molecules including an antisense sequence or oligonucleotide fragment thereof, may be directly introduced into cells of tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes.

As an alternative to full-length antisense BVR mRNA, siRNA can be used to decrease the cellular or nuclear concentration of BVR. siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the BVR nucleotide sequence. siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. siRNA molecules that effectively interfere with BVR expression that are suitable for use in the present invention include, without limitation:

| | |
|---|---|
| 5′-UCCUCAGCGUUCCUGAACCUG; | (SEQ ID NO: 57) |
| and | |
| 3′-AGGAGUCGCAAGGACUUGGAC. | (SEQ ID NO: 58) |

Additional suitable siRNA BVR nucleic acid molecules are disclosed infra in the Examples.

siRNA molecules that effectively interfere with HO-2 expression that are suitable for use in the present invention include, without limitation:

| | |
|---|---|
| 5′-AGGACUUCUUGAAAGGCAACAUUAAAG; | (SEQ ID NO: 59) |
| and | |
| 3′-UAUCCUGAAGAACUUUCCGUUGUAAUU | (SEQ ID NO: 60) |

Other suitable siRNA HO-2 nucleic acid molecules include those disclosed herein in the Examples, and those disclosed by William et al., “Hemoxygenase-2 is an Oxygen Sensor for a Calcium-Sensitive Potassium Channel,” Science 306:2093-97 (2004), which is hereby incorporated by reference in its entirety.

Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway. shRNA molecules that effectively interfere with human BVR expression have been developed and are suitable for use in the methods of the present invention (see e.g., OriGene Technologies (Rockville, Md.) Catalogue No. TR314466).

The various methods and agents for stabilizing or destabilizing intracellular HO-2 levels and activity discussed supra can be used for various therapeutic or prophylactic treatments.

Accordingly, one aspect of the present invention relates to a method of treating a coronary disorder that includes administering to a patient having a coronary disorder an effective amount of an agent that induces the stabilization of HO-2 in cardiac cells, whereby said administering is effective to increase the level of HO-2 in cardiac cells and treat the coronary disorder.

In accordance with this aspect of the present invention, a method of preventing hypoxic damage to cardiac cells and/or preventing cardiac cell death is also provided. This method includes administering to a patient having a coronary disorder characterized by hypoxia or cardiac cell death, or to the cardiac cells directly, an effective amount of an agent that induces the stabilization of HO-2 in cardiac cells, whereby said administering is effective to increase the level of HO-2 in the cardiac cells and protect the cardiac cells against hypoxic damage and cell death caused by the coronary disorder.

As used herein, a “patient” having a coronary disorder or a coronary disorder characterized by hypoxia encompasses any animal, but preferably a mammal. More preferably, the patient is a human.

Coronary disorders that can be treated in accordance with the methods of the present invention can be categorized into at least two groups. Acute coronary disorders result in a sudden blockage of the blood supply to the heart which deprives the heart tissue of oxygen and nutrients, resulting in damage and death of the cardiac tissue. Acute coronary disorders include, for example, myocardial infarction or stroke. In contrast, chronic coronary disorders are characterized by a gradual decrease of oxygen and blood supply to the heart tissue over time causing progressive damage and the eventual death of cardiac tissue. Chronic coronary disorders include chronic coronary ischemia, arteriosclerosis, congestive heart failure, angina, atherosclerosis, and myocardial hypertrophy. Other coronary disorders that can be treated using the methods of the present invention include dilated cardiomyopathy, restenosis, coronary artery disease, arrhythmia, and hypertension.

In accordance with this aspect of the present invention, cardiac cell death or damage in a patient or in vitro is mitigated by increasing the intracellular stabilization and activity of HO-2. The agent administered to the patient having a coronary disorder or to the cardiac cells directly is an agent that enhances the expression of BVR or mimics BVR activity to regulate the stability, expression, and/or activity of HO-2 in cardiac cells. Suitable agents include BVR peptides having an amino acid sequence of KX[C/H][C/H]SXK (SEQ ID NO:7)) and expression systems encoding these BVR peptides.

Another aspect of the present invention relates to a method of inducing cancer cell death in a patient. This method involves administering to a patient having cancer an effective amount of an agent that induces the destabilization of HO-2 in cancer cells of the patient, where said administering is effective to decrease the level of HO-2 in the cancer cells of the patient and thereby induce cancer cell death. In accordance with this aspect of the present invention, the cancer cell can be any type of cancer cell as recited supra.

In one embodiment of the present invention, the agent that induces destabilization of HO-2 is administered directly to the cancer cells of the patient, i.e., direct injection into the tumor site. Alternatively, the agent is administered to the patient systemically, having been formulated to target only cancer cells and not non-cancer cells as described infra.

In accordance with this aspect of the invention, the agent is an inhibitory BVR or HO-2 nucleic acid molecule (i.e., antisense, siRNA, shRNA or miRNA molecule), a BVR peptide having an amino acid sequence of XXX[I/L]LXX (SEQ ID NO:20), XXX[I/L]LXXLXLA (SEQ ID NO:29), FXFPXF[S/T]X (SEQ ID NO:38), or FXXFXFX (SEQ ID NO:47)) as described supra, or a combination thereof.

The mode of affecting delivery and cellular uptake of the agents of the present invention to modulate (i.e., increase or decrease) intracellular HO-2 stabilization and achieve therapeutic utility, vary depending on the type of therapeutic agent (e.g., a BVR peptide, an expression system encoding a BVR peptide, or an inhibitory BVR nucleic acid molecule) being delivered. For example, nucleic acid molecules encoding a BVR peptide or inhibitory BVR nucleic acid molecules may be incorporated into a gene therapy vector to facilitate delivery. Suitable gene therapy vectors include viral expression vectors such as, adenovirus, adeno-associated virus, retrovirus, lentivirus, or herpes virus.

Adenovirus gene delivery vehicles can be readily prepared and utilized given the disclosure provided in Berkner, “Development of Adenovirus Vectors for the Expression of Heterologous Genes,” Biotechniques 6:616-29 (1988); Rosenfeld et al., “Adenovirus-mediated Transfer of a Recombinant α1-Antitrypsin Gene to the Lung Epithelium In Vivo,” Science 252:431-434 (1991), WO 1993/007283 to Curiel et al., WO 1993/006223 to Perricaudet et al., and WO1993/007282 to Curiel et al., which are hereby incorporated by reference in their entirety. Additional types of adenovirus vectors are described in U.S. Pat. No. 6,057,155 to Wickham et al., U.S. Pat. No. 6,033,908 to Bout & Hoeben, U.S. Pat. No. 6,001,557 to Wilson et al., U.S. Pat. No. 5,994,132 to Chamberlain & Kumar-Singh, U.S. Pat. No. 5,981,225 to Kochanek & Schniedner, U.S. Pat. No. 5,885,808 to Spooner & Epenetos, and U.S. Pat. No. 5,871,727 to Curiel, which are hereby incorporated by reference in their entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., “Dual-Target Inhibition of HIV-1 in vitro by Means of an Adeno-associated Virus Antisense Vector,” Science 258:1485-8 (1992), Walsh et al., “Regulated High Level Expression of a Human γ-Globin Gene Introduced into Erythroid Cells by an Adeno-Associated Virus Vector,” Proc Nat'l Acad Sci USA 89:7257-61 (1992), Walsh et al., “Phenotypic Correction of Fanconi Anemia in Human Hematopoietic Cells with a Recombinant Adeno-Associated Virus Vector,” J Clin Invest 94:1440-8 (1994), Flotte et al., “Expression of the Cystic Fibrosis Transmembrane Conductance Regulator from a Novel Adeno-Associated Virus Promoter,” J Biol Chem 268:3781-90 (1993), Ponnazhagan et al., “Suppression of Human α-Globin Gene Expression Mediated by the Recombinant Adeno-Associated Virus 2-based Antisense Vectors,” J Exp Med 179:733-8 (1994), Miller et al., “Recombinant Adeno-Associated Virus (rAAV)-Mediated Expression of a Human γ-Globin Gene in Human Progenitor-Derived Erythroid Cells,” Proc Nat'l Acad Sci USA 91:10183-7 (1994), Einerhand et al., “Regulated High-Level Human β-Globin Gene Expression in Erythroid Cells Following Recombinant Adeno-Associated Virus-Mediated Gene Transfer,” Gene Ther 2:336-43 (1995), Luo et al., “Adeno-Associated Virus 2-Mediated Gene Transfer and Functional Expression of the Human Granulocyte-Macrophage Colony-Stimulating Factor,” Exp Hematol 23:1261-7 (1995), and Zhou et al., “Adeno-Associated Virus 2-Mediated Transduction and Erythroid Cell-Specific Expression of a Human β-Globin Gene,” Gene Ther 3:223-9 (1996), which are hereby incorporated by reference in their entirety. In vivo use of these vehicles is described in Flotte et al., “Stable In Vivo Expression of the Cystic Fibrosis Transmembrane Conductance Regulator with an Adeno-Associated Virus Vector,” Proc Nat'l Acad Sci USA 90:10613-7 (1993), and Kaplitt et al., “Long-Term Gene Expression and Phenotypic Correction Using Adeno-Associated Virus Vectors in the Mammalian Brain,” Nat Genet 8:148-54 (1994), which are hereby incorporated by reference in their entirety.

Retroviral vectors which have been modified to form infective transformation systems can also be used to deliver a recombinant gene encoding a BVR peptide product into a target cell. One such type of retroviral vector is disclosed in U.S. Pat. No. 5,849,586 to Kriegler & Perez, which is hereby incorporated by reference in its entirety. Lentivirus vectors can also be utilized, including those described in U.S. Pat. No. 6,790,657 to Arya, U.S. Patent Application Publication No. 2004/0170962 to Kafri et al., and U.S. Patent Application Publication No. 2004/0147026 to Arya, which are hereby incorporated by reference in their entirety.

Regardless of the type of infective transformation system employed, it should be targeted for delivery of the nucleic acid to a specific cell type. For example, for delivery of the nucleic acid into a cluster of cells (e.g., cardiac cells or cancer cells) a high titer of the infective transformation system can be injected directly within the site of those cells so as to enhance the likelihood of cell infection. The infected cells will then express the desired product, in this case BVR, BVR peptide, or an inhibitory BVR or HO-2 nucleic acid molecule to modify the level of HO-2 present in those cells. The expression system can further contain a promoter to control or regulate the strength and specificity of expression of the therapeutic peptide or nucleic acid molecule in the target tissue or cell.

One of skill in the art can readily select appropriate constitutive mammalian promoters based on their strength as a promoter. As an alternative to constitutive promoters, a mammalian tissue-specific promoter can be utilized. Any of a variety of tissue specific promoters are known in the art and can be selected based upon the tissue or cell type to be treated. When the desired target cell is a cardiac cell, any cardiac cell-specific promoter known in the art, such as the alpha-myosin heavy chain promoter (Akaiwa et al., “Cardiomyocyte-specific Gene Expression Following Recombinant Adeno-associated Viral Vector Transduction,” J. Biol. Chem. 277(21):18979-18985 (2002), which is hereby incorporated by reference in its entirety) or a VSMC-specific promoter such as a synthetic promoter containing the aortic preferentially expressed gene-1 (APEG-1) E box motif (Hsieh et al., “Genomic Cloning and Promoter Analysis of Aortic Preferentially Expressed Gene-1: Identification of a Vascular Smooth Muscle-Specific Promoter Mediated by an E Box Motif,” J Biol Chem 274(20):14344-14351 (1999), which is hereby incorporated by reference in its entirety) can be utilized. Also, a promoter specific for microvascular endothelial cells can be used, such as the promoter of calcitonin receptor-like receptor (CRLR) (Nikitenko et al., “Transcriptional Regulation of the CRLR Gene in Human Microvascular Endothelial Cells by Hypoxia,” FASEB online publication (Jun. 17, 2003), which is hereby incorporated by reference in its entirety).

When the desired target cell is a cancer cell, a cancer cell-specific targeting approach is desirable. Suitable cancer cell-specific targeting approaches include the lentivirus-mediated Tet-On inducible system under the control of the matrix metalloproteinase-2 promoter as described by Seo et al., “Induction of Cancer Cell-Specific Death via MMP2 Promoter-Dependent Bax Expression,” BMB Reports 42(4):217-222 (2009), which is hereby incorporated by reference in it entirety. Also suitable for targeting cancer-specific cells is the dual promoter system described by Fukazawa et al., “Development of a Cancer-Targeted Tissue-Specific Promoter System,” Can. Res. 64:363-369 (2004), which is hereby incorporated by reference in its entirety, that combines the human telomerase reverse transcriptase promoter (hTERT) and a tissue specific promoter (e.g., human surfactant protein A1 promoter for directing lung cell specific expression or prostate-specific antigen (PSA) for colon cancer cell specific expression) to target expression to cancer cells. Other cancer cell-specific targeting approaches utilizing the hTERT tumor-specific promoter are also suitable for use in the present invention (see, e.g., Fang et al., “Development of Chimeric Gene Regulators for Cancer-Specific Gene Therapy with Both Transcriptional and Translational Targeting,” Mol. Biotechnol. 45:71-81 (2010), Gu et al., “Tumor-Specific Transgene Expression from the Human Telomerase Reverse Transcriptase Promoter Enables Targeting of the Therapeutic Effects of the Bax Gene to Cancers,” Can. Res. 60:5359-64 (2000), and Gu et al., “A Novel Single Tetracycline-Regulative Adenoviral Vector for Tumor-Specific Bax Gene Expression and Cell Killing In Vitro and In Vivo,” Oncogene 21:4757-62 (2002), which are hereby incorporated by reference in their entirety.

Gene expression can be regulated to achieve optimal expression levels and reduce side effects associated with constitutive gene expression. Whether the promoter is tissue-specific or not, the promoter can also be made inducible for purposes of controlling when expression or suppression of the BVR protein or peptide fragment is desired. One of skill in the art can readily select appropriate inducible mammalian promoters from those known in the art. One exemplary inducible promoter includes a Tet-O response element (Farson et al., “A New-Generation Stable Inducible Packaging Cell Line for Lentiviral Vectors,” Hum. Gene Ther 12(8):981-97 (2001), which is hereby incorporated by reference in its entirety). When used in combination with a tissue-specific promoter, the Tet-O response elements can render a tissue-specific promoter inducible to tetracycline and its derivatives (see e.g., Michalon et al., “Inducible and Neuron-Specific Gene Expression in the Adult Mouse Brain with the rtTA2S-M2 System,” Genesis 43(4):205-12 (2005), which is hereby incorporated by reference in its entirety).

Another approach that is appropriate for cardiac cell protection against ischemia/reperfusion injury may involve turning on gene expression with the onset of ischemia (hypoxia), so that the gene product is already present during reperfusion. Many transcription factors are modified by hypoxic and oxidative stress. Studies of molecular responses to hypoxia have identified HIF-1α as the master regulator of hypoxia-inducible gene expression. Under hypoxic conditions, HIF-1α binds to the hypoxia-responsive element (HRE) in the enhancer region of its target genes and turns on gene transcription. Additionally, reperfusion or reoxygenation after ischemia increases the transactivating ability of NFκB. Genes regulated by NFκB include cytokines and adhesion molecules, which contribute to cell death by promoting inflammatory responses. Several studies indicate that the hypoxic and hyperoxic environment can be used to activate heterologous gene expression driven by HRE and cis-acting consensus sequences of activated NFκB respectively. Accordingly, in one aspect of the invention, at least one HRE is utilized as an enhancer to drive transgene expression in the expression system encoding the desired BVR protein or peptide sequence. Suitable HRE nucleic acid constructs and expression systems are described in U.S. Pat. No. 5,942,434 to Ratcliffe et al., which is hereby incorporated by reference in its entirety. To assure sufficient duration of the transgene expression to achieve myocardial protection during the reperfusion period, a second regulatory element that is activated by oxidative stress such as NFκB responsive element is employed.

Recombinant cell therapy, which is a form of gene therapy, can also be utilized for the delivery of nucleic acid molecule encoding the desired BVR peptides to cardiac tissue for the treatment of coronary disorders. In this approach, a target cell is transfected (i.e., transformed or transduced) with a nucleic acid encoding the BVR protein or modulating peptide and, in turn, the target cell produces the gene product that exerts a therapeutic effect, e.g., inhibition of cell damage such as cardiomyocyte death after a hypoxia-related injury. Target cell transfection can be transient or stable (i.e., the nucleic acid becomes integrated into the genome of the target cell), and the target cell can be heterologous, or preferably autologous to the patient. Once the target cell or cells are producing the BVR protein or modulating peptide, they are administered to the patient as described below.

This approach is preferably carried out using recombinant mesenchymal stem cells (MSCs) expressing a recombinant nucleic acid encoding BVR or modulating BVR peptide operably linked to a promoter, preferably a tissue-specific promoter of the type described above. MSCs are multi-potent progenitor cells known to have a broad potential for cellular differentiation into more than one type of cell lineage and have a greatly reduced incidence of immune system-mediated rejection when grafted into non-autologous hosts. MSCs have a demonstrated ability to differentiate into cardiomyocytes, vascular endothelial and connective tissue (see e.g., Pittenger et al., “Multilineage Potential of Adult Human Mesenchymal Stem Cells,” Science 284:143-147 (1999); U.S. Pat. Nos. 6,387,369 to Pittenger et al., 6,214,369 to Grande et al., 5,906,934, and 5,827,735 to Grande et al., which are hereby incorporated by reference in their entirety.

Delivery of the recombinant MSCs (rMSCs) into the heart of a patient may be either direct (i.e., injection in vivo of rMSCs to a patient's cardiac tissue) or indirect (i.e., perfusion of rMSCs into the peripheral blood vessel of a subject, with subsequent homing of the rMSC to the injured cardiac tissue). In some embodiments, 5×10⁶ rMSCs are injected into the treatment site. The number of rMSCs injected per treatment site can range from at least 1×10⁴ cells to at least 1×10⁸ cells.

Therapeutic BVR peptides or nucleic acid molecule encoding the peptides can also be delivered to the target cell (i.e., cardiac cells or cancer cells) using liposomes. Basically, this involves providing a liposome which encapsulates the BVR peptide or nucleic acid to be delivered, and then contacting the target cell with the liposome under conditions effective for delivery of the peptide or nucleic acid into the cell.

Liposomes are vesicles comprised of one or more concentrically ordered lipid bilayers which encapsulate an aqueous phase. They are normally not leaky, but becomes leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug, in this case the BVR peptide or nucleic acid at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be regulated.

In contrast to passive drug release, active drug release involves using an agent to induce a permeability change in the liposome vesicle. Liposome membranes can be constructed so that they become destabilized when the environment becomes acidic near the liposome membrane (see e.g., Wang et al., “pH-Sensitive Immunoliposomes Mediate Target-Cell-Specific Delivery and Controlled Expression of a Foreign Gene in Mouse,” Proc Natl Acad Sci USA 84:7851 (1987), which is hereby incorporated by reference in its entirety). When liposomes are endocytosed by a target cell, for example, they can be routed to acidic endosomes which will destabilize the liposome and result in drug release. Alternatively, the liposome membrane can be chemically modified such that an enzyme is placed as a coating on the membrane which slowly destabilizes the liposome. The liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J Mol Biol 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference in their entirety.

In a preferred embodiment of the present invention, the liposome is conjugated to an antibody targeting the liposome to a desired target cell, in this case a cardiac cell or cancer cell. For example, to target the lipsome to a cancer cell the liposome may be conjugated to an anti-C3B(I) antibody as disclosed by U.S. Pat. No. 6,572,856 to Taylor et al., an alphafeto protein receptor as disclosed by U.S. Pat. No. 6,514,685 to Moro, or the monoclonal GAH antibody as disclosed by U.S. Pat. No. 5,837,845 to Hosokawa, which are all hereby incorporated by reference in their entirety. For targeting the liposome to cardiac cells, the lipsome may be conjugated to an antibody recognizing elastin microfibril interfacer (EMILIN2) (Van Hoof et al., “Identification of Cell Surface for Antibody-Based Selection of Human Embryonic Stem Cell-Derived Cardiomyocytes,” J Proteom Res 9:1610-18 (2010), cardiac troponin I, connexin-43, or any cardiac cell-surface membrane receptor that is known in the art.

Like liposomes, micelles have also been used in the art for drug delivery. A number of different micelle formulations have been described in the literature for use in delivery proteins or polypeptides, and others have been described which are suitable for delivery of nucleic acids. Any suitable micelle formulations can be adapted for delivery of the therapeutic protein, peptide, or nucleic acid of the present invention. Exemplary micelles include without limitation those described, e.g., in U.S. Pat. No. 6,210,717 to Choi et al.; and U.S. Pat. No. 6,835,718 to Kosak, each of which is hereby incorporated by reference in its entirety.

An alternative approach for delivery of peptides or nucleic acids involves the conjugation of the desired therapeutic agent to a polymer that is stabilized to avoid enzymatic degradation of the conjugated protein or polypeptide. Conjugated proteins or peptides of this type are described in U.S. Pat. No. 5,681,811 to Ekwuribe, which is hereby incorporated by reference in its entirety. Nucleic acid conjugates are described in U.S. Pat. No. 6,528,631 to Cook et al., U.S. Pat. No. 6,335,434 to Guzaev et al., U.S. Pat. No. 6,235,886 to Manoharan et al., U.S. Pat. No. 6,153,737 to Manoharan et al., U.S. Pat. No. 5,214,136 to Lin et al., or U.S. Pat. No. 5,138,045 to Cook et al., each of which is hereby incorporated by reference in its entirety.

Yet another approach for delivery of peptides of the invention involves preparation of chimeric targeting peptides according to U.S. Pat. No. 5,817,789 to Heartlein et al., which is hereby incorporated by reference in its entirety. The chimeric peptide includes a ligand domain and the BVR peptide. The ligand domain is specific for a cardiac cell or vascular endothelial cell receptor. Alternatively, the ligand domain is specific for a cancer cell. For example, U.S. Pat. No. 5,679,350 to Jankun et al., which is hereby incorporated by reference in its entirety, discloses methods of coupling cancer therapeutics to plasminogen activator inhibitor type-1 or type-2 to target delivery to cancer cells. Thus, when the chimeric peptide is delivered intravenously or otherwise introduced into blood or lymph, the chimeric peptide will selectively bind to the target cell, and the target cell will internalize the chimeric peptide.

Another aspect of the present invention relates to isolated BVR peptides, including any of the BVR peptides described above, and pharmaceutical compositions containing an isolated BVR peptide of the invention and a pharmaceutical carrier.

The isolated peptides of the present invention may be prepared using standard methods of synthesis known in the art, including solid phase peptide synthesis (Fmoc or Boc strategies) or solution phase peptide synthesis. Alternatively, peptides of the present invention may be prepared using recombinant expression systems.

Generally, the use of recombinant expression systems involves inserting the nucleic acid molecule encoding the amino acid sequence of the desired peptide into an expression system to which the molecule is heterologous (i.e., not normally present). One or more desired nucleic acid molecules encoding a peptide of the invention may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

The preparation of the nucleic acid constructs can be carried out using standard cloning procedures well known in the art as described by Joseph Sambrook et al., MOLECULAR CLONING: A LABORATORY MANUAL (Cold Springs Harbor 1989). U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in a suitable host cell.

A variety of genetic signals and processing events that control many levels of gene expression (e.g., DNA transcription and messenger RNA (“mRNA”) translation) can be incorporated into the nucleic acid construct to maximize peptide production. For the purposes of expressing a cloned nucleic acid sequence encoding a desired peptide, it is advantageous to use strong promoters to obtain a high level of transcription. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Common promoters suitable for directing expression in mammalian cells include, without limitation, SV40, MMTV, metallothionein-1, adenovirus E1a, CMV, immediate early, immunoglobulin heavy chain promoter and enhancer, and RSV-LTR.

There are other specific initiation signals required for efficient gene transcription and translation in prokaryotic cells that can be included in the nucleic acid construct to maximize peptide production. Depending on the vector system and host utilized, any number of suitable transcription and/or translation elements, including constitutive, inducible, and repressible promoters, as well as minimal 5′ promoter elements, enhancers or leader sequences may be used. For a review on maximizing gene expression see Roberts and Lauer, “Maximizing Gene Expression On a Plasmid Using Recombination In Vitro,” Methods in Enzymology 68:473-82 (1979), which is hereby incorporated by reference in its entirety.

A nucleic acid molecule encoding an isolated peptide of the present invention, a promoter molecule of choice, including, without limitation, enhancers, and leader sequences, a suitable 3′ regulatory region to allow transcription in the host, and any additional desired components, such as reporter or marker genes, are cloned into the vector of choice using standard cloning procedures in the art, such as described in Joseph Sambrook et al., MOLECULAR CLONING: A LABORATORY MANUAL (Cold Springs Harbor 1989); Frederick M. Ausubel, SHORT PROTOCOLS IN MOLECULAR BIOLOGY (Wiley 1999), and U.S. Pat. No. 4,237,224 to Cohen and Boyer, which are hereby incorporated by reference in their entirety.

Once the nucleic acid molecule encoding the peptide has been cloned into an expression vector, it is ready to be incorporated into a host. Recombinant molecules can be introduced into cells, without limitation, via transfection (if the host is a eukaryote), transduction, conjugation, mobilization, or electroporation, lipofection, protoplast fusion, mobilization, or particle bombardment, using standard cloning procedures known in the art, as described by JOSEPH SAMBROOK et al., MOLECULAR CLONING: A LABORATORY MANUAL (Cold Springs Harbor 1989), which is hereby incorporated by reference in its entirety.

A variety of suitable host-vector systems may be utilized to express the recombinant protein or polypeptide. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria.

Purification of peptide produced via recombinant methods may be achieved by several methods readily known in the art, including ion exchange chromatography, hydrophobic interaction chromatography, affinity chromatography, gel filtration, and reverse phase chromatography. The peptide is preferably produced in purified form (preferably at least about 80% or 85% pure, more preferably at least about 90% or 95% pure) by conventional techniques. Depending on whether the recombinant host cell is made to secrete the peptide into growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide is subjected to gel filtration in an appropriately sized dextran or polyacrylamide column to separate the peptides from other proteins. If necessary, the peptide fraction may be further purified by HPLC.

Pharmaceutical compositions containing the isolated BVR peptides of the present invention also contain pharmaceutically or physiologically acceptable carriers, excipients, or stabilizers, or in solid or liquid form such as, tablets, capsules, powders, solutions, suspensions, or emulsions, they can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery. For most therapeutic purposes, peptides or nucleic acids can be administered intravenously.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the peptides or nucleic acids in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Other delivery systems that are known in the art for cardiac delivery or delivery to cancer cells can be modified for delivery of the therapeutic proteins, peptides, or nucleic acid molecules described supra.

### EXAMPLES

The following examples illustrate various methods for compositions in the treatment method of the invention. The examples are intended to illustrate, but in no way limit, the scope of the invention.

### Materials and Methods for Examples 1-6

Neonatal Rat Cardiomyocytes: Primary cultures of neonatal rat cardiomyocytes were prepared as described previously (Ding et al., “Functional Role of Phosphodiesterase 3 in Cardiomyocyte Apoptosis: Implication in Heart Failure,” Circulation 111:2469-2476 (2005) which is hereby incorporated by reference in its entirety). Briefly, fragmented ventricular tissue from 1-2 day old Sprague-Dawley rats was subjected to multiple rounds of enzymatic digestion with collagenase II (Worthington, Lakewood, N.J.). The cells were then collected by low speed centrifugation at 4° C. Non-myocytes were removed by two rounds of pre-plating the cells on culture dishes. The enriched cardiomyocytes were cultured in DMEM with 10% FBS and 10% horse serum. The day after cell adhesion, 10 μM cytosine 1-β-D-arabinofuranoside (Sigma, St. Louis, Mo.) was added to inhibit the growth of contaminating non-myocytes. Cells were treated with 10 mM (−)-isoproterenol (Sigma) for the times indicated in the figures.

Plasmids and Constructs: Adenovirus for expression of an endogenous PKA inhibitor (PKA251 bp) was prepared as described previously (Ding et al., “Functional Role of Phosphodiesterase 3 in Cardiomyocyte Apoptosis: Implication in Heart Failure,” Circulation 111:2469-2476 (2005), which is hereby incorporated by reference in its entirety). Rat BVR and HO-2 siRNA were designed based on the human sequences (Miralem et al., “Small Interference RNA-Mediated Gene Silencing of Human Biliverdin Reductase, but not that of Heme Oxygenase-1, Attenuates Arsenite-Mediated Induction of the Oxygenase and Increases Apoptosis in 293A Kidney Cells,” J Biol Chem 280:17084-17092 (2005), which is hereby incorporated by reference in its entirety). The BVR targeting site is at nucleotides 94-114, and the HO-2 targeting site is at nucleotides 73-93, downstream of the start codon in each case. Oligonucleotides containing the sequence of a 21 nucleotide small interference RNA were synthesized as follows: BVR siRNA, 5′-GATCCCC (TCTGCAGCATTCCTGAACCTG) TTCAAGAG (ACAGGTTCAGGAATGCTGCAGA) TTTTTGGAAA (SEQ ID NO:61) and 5′-AGCTTTTCCAAAAA (TCTGCAGCATTCCTGAACCTG) TCTCTTGAA (CAGGTTCAGGAATGCTGCAGA) GGG (SEQ ID NO:62; HO-2 siRNA, 5′-GATCCCC (GAAAACCATACCAAAATGGCA) TTCAAGAGA (TGCCATTTTGGTATGGTTTTC) TTTTTGGAAA (SEQ ID NO:63) and 3′-AGCTTTTCCAAAAA (GAAAACCATACCAAAATGGCA) TCTCTTGAA (TGCCATTTTGGTATGGTTTTC) GGG (SEQ ID NO:64). Complementary oligonucleotides were annealed, then ligated into pSuper-Retro vector (OligoEngine Co. Seattle, Wash.). E. coli clones containing the desired constructs were identified by restriction analysis and verified by DNA sequencing. The retroviral DNA vectors for generating BVR si-rRNA and HO-1 si-rRNA were transfected into the HEK293A packaging cell line, and the supernatant containing the expressed si-rBVR or si-rHO-2 retrovirus was then titrated in NIH3T3 cells. Retrovirus expressing siRNA was used to infect cells at a multiplicity of infection of 4 plaque-forming units/cell. The plasmid pcDNA-hBVR has previously been described (Lerner-Marmarosh et al., “Human Biliverdin Reductase: A Member of the Insulin Receptor Substrate Family with Serine/Threonine/Tyrosine Kinase Activity,” Proc Natl Acad Sci US A 102:7109-7114 (2005), which is hereby incorporated by reference in its entirety); pcDNA-hHO2 was constructed by cloning the HO-2 open reading frame into pcDNA3.

Cell Culture and Transfection of HEK293A Cells with hBVR and hHO-2 Expression Vectors and si-rBVR: The human embryonic kidney cell line HEK293A was obtained from Invitrogen, (Carlsbad, Calif.). Cells were grown in 10 cm plates with Dulbecco's modified Eagle's medium containing 10% fetal bovine serum and penicillin-G/streptomycin for 24 hours or until 70% confluency was reached. Cells were subsequently transfected with 4 μg of plasmid in each 10 cm dish using Transfectin reagent (Bio-Rad, Hercules, Calif.), according to the manufacturer's protocol. Twenty-four hours after DNA addition, and if appropriate, infection with culture supernatant containing retrovirus expressing si-hBVR (at a multiplicity of infection of 30) and cycloheximide (10 μg/ml) was added, and samples were removed atthe appropriate times for protein analysis. Additional si-RNA virus was added every 12 hours during the experiment.

Western Blot Analysis: Cell lysates were prepared by homogenization in RIPA buffer (40 mM Tris-HCl, pH 7.5, 1 mM EDTA, 1 mM DTT, 0.1 mM Na₃VO₄, 10 μg/ml aprotinin, 5 μg/ml pepstatin, 20 μg/ml leupeptin, 1 mM bezamidine followed by centrifugation at 800×g for 10 minutes at 4° C. (Ding et al., “Functional Role of Phosphodiesterase 3 in Cardiomyocyte Apoptosis: Implication in Heart Failure,” Circulation 111:2469-2476 (2005), which is hereby incorporated by reference in its entirety). Supernatant proteins were resolved by SDS-PAGE and transferred to nitrocellulose. Western blots were examined using antibodies against β-actin (Cell Signaling), BVR (Huang et al., “Detection of 10 Variants of Biliverdin Reductase in Rat Liver by Two-Dimensional Gel Electrophoresis,” J Biol Chem 264:7844-7849 (1989), which is hereby incorporated by reference in its entirety), HO-1, HO-2 (Panahian et al., “Site of Injury-Directed Induction of Heme Oxygenase-1 and -2 in Experimental Spinal Cord Injury Differential Functions in Neuronal Defense Mechanisms?” J Neurochem 76:539-554 (2001), which is hereby incorporated by reference in its entirety), and cleaved caspase-3 (Cell Signaling, Beverly Mass.). The secondary antibody was horseradish peroxidase-conjugated goat anti-rabbit IgG (1:2,000, Bio-Rad).

Quantitative RT-PCR: Total RNA was isolated using Trizol reagent (Invitrogen). The first-strand cDNA synthesis and quantitative polymerase chain reaction (RT-PCR) were performed as previously described (Tudor et al., “Biliverdin Reductase is a Transporter of Haem into the Nucleus and is Essential for Regulation of HO-1 Gene Expression by Haematin,” Biochem J 413:405-416 (2008), which is hereby incorporated by reference in its entirety). The following primers were used for PCR analysis:

GAPDH,
(SEQ ID NO: 65)
5′-TGATGCTGGTGCTGAGTATGTCGT (antisense),
(SEQ ID NO: 66)
5′-TTGTCATTGAGAGCAATGCCAGCC (sense);
BVR,
(SEQ ID NO: 67)
5′-TGCCGAGCCAAAGAGGAAATTTGG (sense);
(SEQ ID NO: 68)
5′-AGCTGTGAAGCGAAGAGACCCTTT (antisense);
HO-1,
(SEQ ID NO: 69)
5′TTAAGCTGGTGATGGCCTCCTTGT (sense),
(SEQ ID NO: 70)
5′-CATGGCCTTCTGCGCAATCTTCT (antisense);
and
HO-2
(SEQ ID NO: 71)
5′-AATGGCAGACCTTTCTGAGCTCCT (sense),
(SEQ ID NO: 72)
5′-GGGCATTGTCCACATGCTCAAACA (antisense).

PCR products were resolved by electrophoresis and quantified by image analysis of stained gels.

Analysis of Apoptosis: The terminal deoxyribonucleotide transferase-mediated dUTP nick-end labeling (TUNEL) assay was used to detect in situ DNA fragmentation, using the In Situ Cell Death Detection Kit (Roche) as described previously (Ding et al., “Functional Role of Phosphodiesterase 3 in Cardiomyocyte Apoptosis: Implication in Heart Failure,” Circulation 111:2469-2476 (2005), which are hereby incorporated by reference in their entirety). In addition, the cells were stained for cardiomyocyte-specific sarcomeric α-actinin with the antibody EF-53 to distinguish cardiomyocytes from contaminating fibroblasts; and only EF-53 positive cells were counted. Three independent experiments were performed and an average of 1,000 EF-53 positive cells from random fields were analyzed.

DNA Electrophoresis: Total cellular DNA was isolated from untreated and treated cells by lysis in 10 mM Tris-HCl, pH 7.8; 1 mM, EDTA, 10 mM NaCl, 1% (w/v) SDS and 1 mg/ml proteinase K at 60° C. for 2 hours. The lysate was extracted twice each with phenol-choloroform (1:1, v/v) and chloroform, followed by precipitation with ethanol. The precipitate was collected by centrifugation, washed once in 70% ethanol, and resuspended in TE containing 1 mg/ml RNase at 37° C. for 30 min. DNA was resolved by electrophoresis in 2.0% agarose gels containing ethidium bromide.

In Vivo Experimental Langendorff Model: Male Sprague-Dawley rats weighing 250-300 g (Charles River, Boston, Mass.) were used in this study; the protocol is based on that described by Tompkins et al., “Mitochondrial Dysfunction in Cardiac Ischemia-Reperfusion Injury: ROS From Complex I, Without Inhibition,” Biochim Biophys Acta 1762:223-231 (2006), which is hereby incorporated by reference in its entirety, with minor modification. The rats were fed a standard rat chow and had water ad libitum. All experiments were approved by the University of Rochester Institutional Animal Care and Use Committee. Rats were given two subcutaneous injections of isoproterenol (ISO) (0.01 mg/kg), 6 hours apart, then anesthetized with freshly prepared Avertin (2,2,2-tribromoethanol, 0.5 mg/kg injected intraperitoneally), and treated with heparin (5000 U/kg), i.p., to protect the heart against microthrombi. The aorta was cannulated in situ with a 22-gauge needle filled with warm Krebs-Henseleit (KH) buffer. The heart was rapidly transferred (<10 s.) to a perfusion apparatus, and retrograde (Langendorff) perfusion begun with 37° C. KH buffer gassed with 95% O₂ plus 5% CO₂, in constant flow mode (12 ml/min). Perfusion was performed using 0.1 μM ISO or KH buffer as a control; the hBVR-derived peptides KKRILHC²⁸¹ (25 μM) or KYCCSRK²⁹⁶ (25 μM) were included in perfusion medium with or without ISO. Hemodynamics and heart rate were recorded by inserting a water-filled balloon connected to a transducer (Radnoti LLC, model no. 159904B) into the left ventricle. The transducer was connected to a data acquisition system (DATAQ Instrument, Akron Ohio, Model: DI-205). At the end of each experiment, the heart was cut into 5 μm sections for TUNEL study or homogenized for western blots.

Implanting ISO Miniosmotic Pump: Miniosmotic pumps (Alzet model 2001, Durect Co., Cupertino, Calif.) were filled under sterile conditions with ISO dissolved in 0.001 N HCl, primed in sterile saline at 37° C. overnight, and aseptically implanted subcutaneously under halothane anesthesia. The ISO concentration was calculated to allow the pumps to deliver the drug at an infusion rate of 400 μg ISO base/kg/h, essentially as described by (Hayes et al., “Effects of Prolonged Isoproterenol Infusion on Cardiac and Vascular Responses to Adrenoceptor Agonists,”J Pharmacol Exp Ther 237:757-763 (1986), which is hereby incorporated by reference in its entirety).

Measurement of BVR Activity: Cardiomyocytes and heart tissue samples were lysed in buffer containing 100 mM sodium phosphate pH 7.4, 1% Nonidet-P-40, 10% glycerol, 0.2 mM dithiothoeitol, 10 mM NaF, protease inhibitor mixture (10 μg/ml aprotinin, 10 μg/ml leupeptin, 10 μg/ml pepstatin, and 0.1 mM phenylmethylsulfonyl fluoride). BVR activity was measured at pH 6.7 as described previously (Huang et al., “Detection of 10 Variants of Biliverdin Reductase in Rat Liver by Two-Dimensional Gel Electrophoresis,”J Biol Chem 264:7844-7849 (1989), which is hereby incorporated by reference in its entirety). The rate of conversion of biliverdin to bilirubin was determined as the increase in absorbance at 450 nm at 25° C. Specific activity is expressed as nanomoles of bilirubin/min/mg of protein.

### Example 1

### Isoproterenol Increases BVR and HO-2 Protein Expression In Vitro

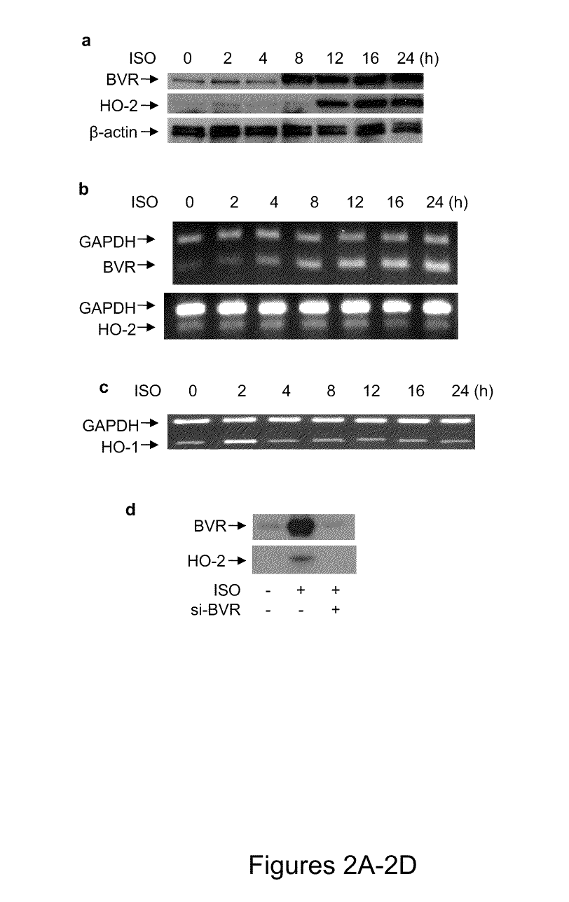

Isolated rat neonatal cardiomyocytes were initially treated with 10 μM isoproterenol. BVR protein expression was elevated within 8 hours of initiating the treatment, while the HO-2 protein level began to increase by 12 hours, reaching a plateau by 24 h (FIG. 2A). The increased levels of both proteins remained stable for up to 96 hours of treatment with isoproterenol.

The question of whether the increased synthesis of each protein required elevated levels of BVR and HO-2 mRNA was investigated by RT-PCR. Interestingly, it was found that the level of BVR mRNA increased in advance of the increased protein synthesis, but the HO-2 mRNA content remained unchanged during isoproterenol stimulation (FIG. 2B). The BVR mRNA content of the cells remained high for up to four days—even prolonged exposure of the cells to isoproterenol did not alter the levels of HO-2 mRNA. Other than a transient increase in the level of HO-1 mRNA (FIG. 2C), HO-1 mRNA remained effectively constant throughout the experiment. HO-1 protein expression was not detected throughout the whole process of isoproterenol treatment up to 96 hours.

The foregoing suggests that increased BVR levels reflect, and are dependent on, the availability of elevated mRNA, while a mechanism that is independent of transcription is responsible for the enhanced levels of HO-2. To test the requirement for BVR mRNA, cardiomyocytes were treated with virus expressing si-rBVR, followed 12 hours later by 10 μM isoproterenol treatment for 24 hours. In untreated cells (FIG. 2D), isoproterenol caused the expected induction of BVR, whereas this was efficiently blocked by si-rBVR treatment. Interestingly, si-rBVR treatment also effectively blocked the induction of HO-2. Thus the observed, time-dependent increase in HO-2 (FIG. 2A) is dependent on the prior induction of BVR.

### Example 2

### Isoproterenol Increases BVR and HO-2 Expression in the Rat Heart During Isoproterenol Infusion

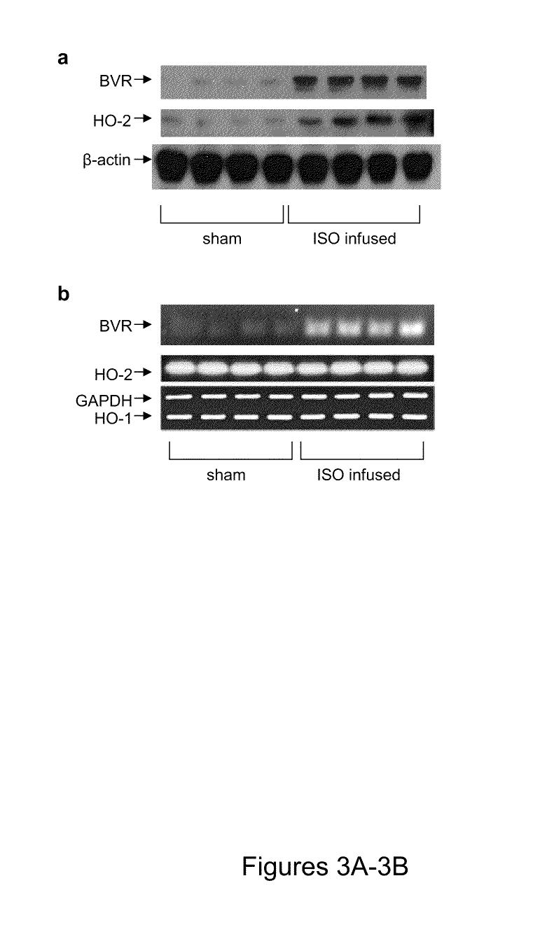

It might be argued that the above data arose as an artifact of cell culture. To test whether those findings also apply in vivo, four rats were continuously infused with isoproterenol for four days, after which cardiac tissue was harvested for examination of BVR and HO-2 expression. Again, the HO-2 and BVR protein expressions were up-regulated compared to those seen in sham treated animals (FIG. 3A). These data are consistent with the in vitro observations. Also, as expected from in vitro observations, there was no change in the level of HO-2 mRNA during infusion with isoproterenol (FIG. 3B), whereas the BVR mRNA was increased. Again, there was no apparent change in the level of HO-1 mRNA in the tissue.

### Example 3

### Induction of BVR and HO-2 is Mediated by the cAMP-PKA Pathway

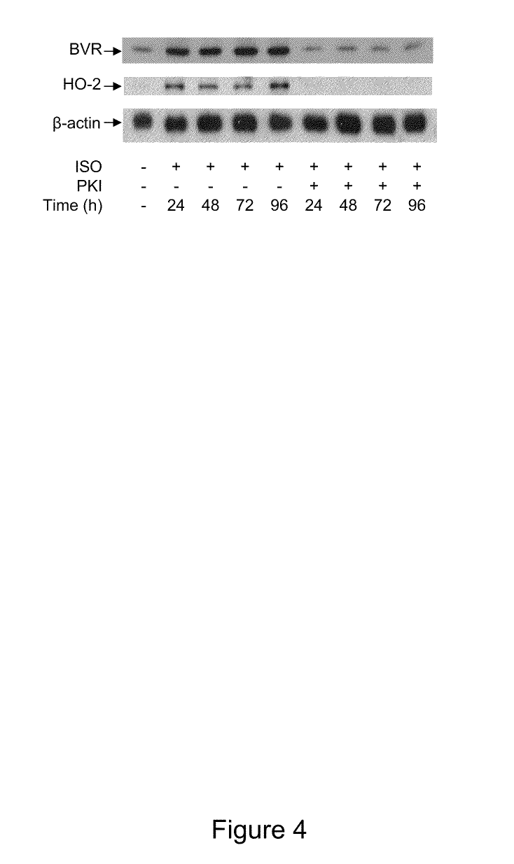

Because the effects of isoproterenol occur via the cAMP-PKA pathway, the effect of inhibiting PKA was tested on the induction of BVR and HO-2 proteins in cultured cardiomyocytes. Adenovirus-mediated expression of the 75-amino acid endogenous PKA inhibitor, PKI, blocked the isoproterenol-induced up-regulation of both BVR and HO-2 (FIG. 4). Thus, the increased levels of both BVR and HO-2 protein after isoproterenol treatment are dependent on the activity of the cAMP-PKA pathway. Because both proteins are regulated by the same pathway, and this pathway first results in induction of BVR, with HO-2 induction being dependent on BVR the following Examples demonstration how elevated BVR cause enhanced HO-2 expression.

### Example 4

### Blockage of BVR Protein Expression Inhibits the HO-2 Protein Induction but not Vice Versa

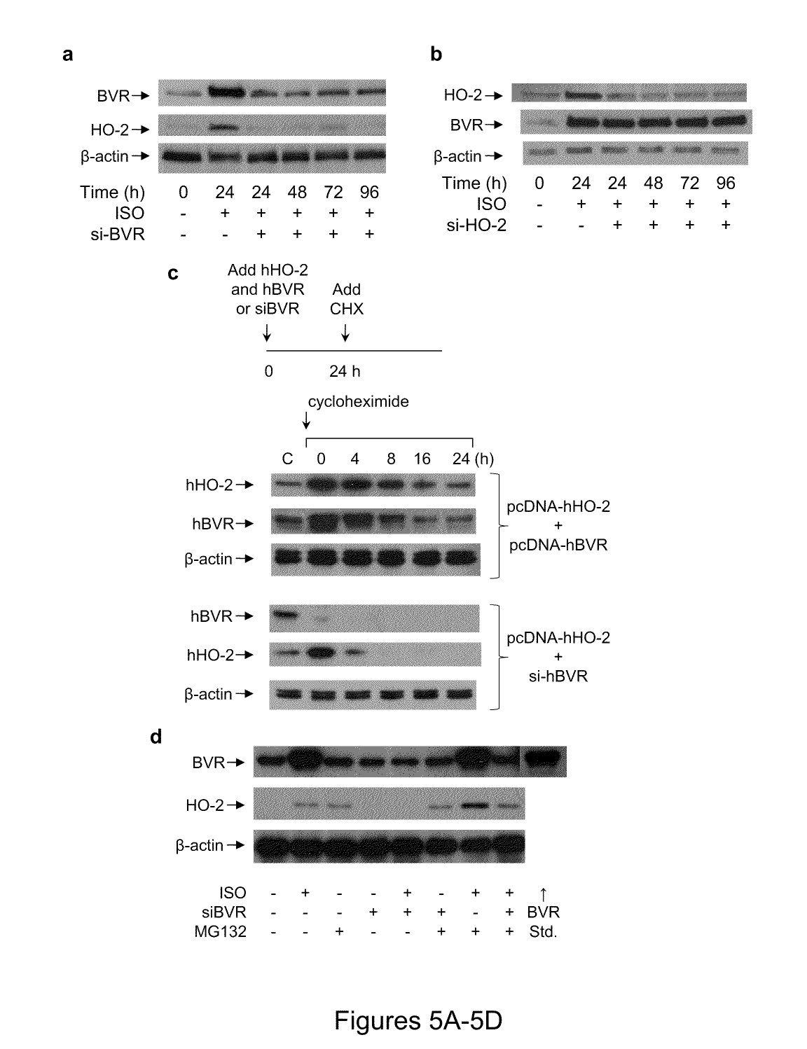

To test further whether expression of HO-2 and BVR proteins are dependent on one another, siRNAs (si-rBVR, si-rHO-2) were used to inhibit the expression of one protein, after which the expression of both proteins were assayed. As expected from the data of FIG. 2D, when retroviral si-rBVR was used to infect rat neonatal cardiomyocytes, the expression of BVR was blocked, even after stimulation by isoproterenol, and moreover, the expression of HO-2 protein was also blocked (FIG. 5A). In the converse experiment, inhibition of HO-2 protein expression with si-rHO-2 had no effect on the BVR protein level (FIG. 5B), although, as expected, HO-2 expression was ablated. These experiments confirm that the increase in HO-2 protein expression after stimulation by isoproterenol is dependent on prior induction of BVR, which by definition occurs independently of HO-2. Based on this observation, further exploration was made concerning the mechanism mediating the interaction of HO-2 and BVR.

### Example 5

### Isoproterenol Stimulation of BVR Levels can Enhance the Stability of HO-2

It is apparent that the level of HO-2 protein observed in isoproterenol-stimulated cardiomyocytes increases in the absence of increased HO-2 mRNA synthesis (FIG. 2B). The regulation of protein levels in the cell is dependent upon at least two factors: synthesis, which is broadly correlated with the mRNA level, and degradation by either of two main pathways, the lysosome or by ubiquitination and subsequent targeting to the proteasome. The elevated HO-2 protein level in the absence of increased mRNA suggests that there is little likelihood of an increased rate of protein synthesis, although this cannot entirely be ruled out—a shift in distribution between the poorly translated 1.9 kb HO-2 mRNA and the well translated 1.3 kb species (Sun et al., “Heme Oxygenase-2 mRNA: Developmental Expression in the Rat Liver and Response To Cobalt Chloride,” Arch Biochem Biophys 282:340-345 (1990), which is hereby incorporated by reference in its entirety) might lead to the observed result. The more likely possibility is altered protein degradation, and it is believed that the enhanced HO-2 protein level is due to increased protein stability. Two experiments were designed to examine the dependency of HO-2 protein stability on the level of BVR protein. As shown in FIG. 5C, one set of HEK293A cells was cotransfected with pcDNA-hBVR and pcDNA-hHO-2, while a second set was transfected with pcDNA-hHO-2 and concomitantly infected with retrovirus expressing si-hBVR. Twenty-four hours later, the cells were treated with the protein synthesis inhibitor cycloheximide for up to 24 hours. In those cells over-expressing BVR, HO-2 decayed relatively slowly, demonstrating a half-life of ˜8 hours, similar to that of BVR. In cells where BVR expression was ablated, however, hHO-2 was highly unstable, with little remaining at 4 hours; decay was essentially complete at 8 hours. Thus, the stability of HO-2 is critically dependent upon the availability of BVR. The enhanced BVR-dependent stability of HO-2, due to isoproterenol stimulation, could be a consequence of reduced ubiquitination of HO-2; this mechanism was tested by using the proteasome inhibitor MG132 to block ubiquitin-dependent degradation, and examination of the BVR and HO-2 protein levels (FIG. 5D). Interestingly, it was found that the HO-2 protein level was increased in MG132-treated cells in the absence of isoproterenol stimulation. Moreover, MG132 increased the HO-2 protein levels, even if BVR gene expression was inhibited by si-BVR. These results suggest that the isoproterenol-stimulated increase in BVR protein promotes the stability of HO-2, and that this is mediated by the inhibition of proteosomal degradation of presumably ubiquitinated HO-2.

### Example 6

### The Interaction Between HO-2 and BVR is a Survival Factor for Cardiomyocytes During Isoproterenol Stimulation

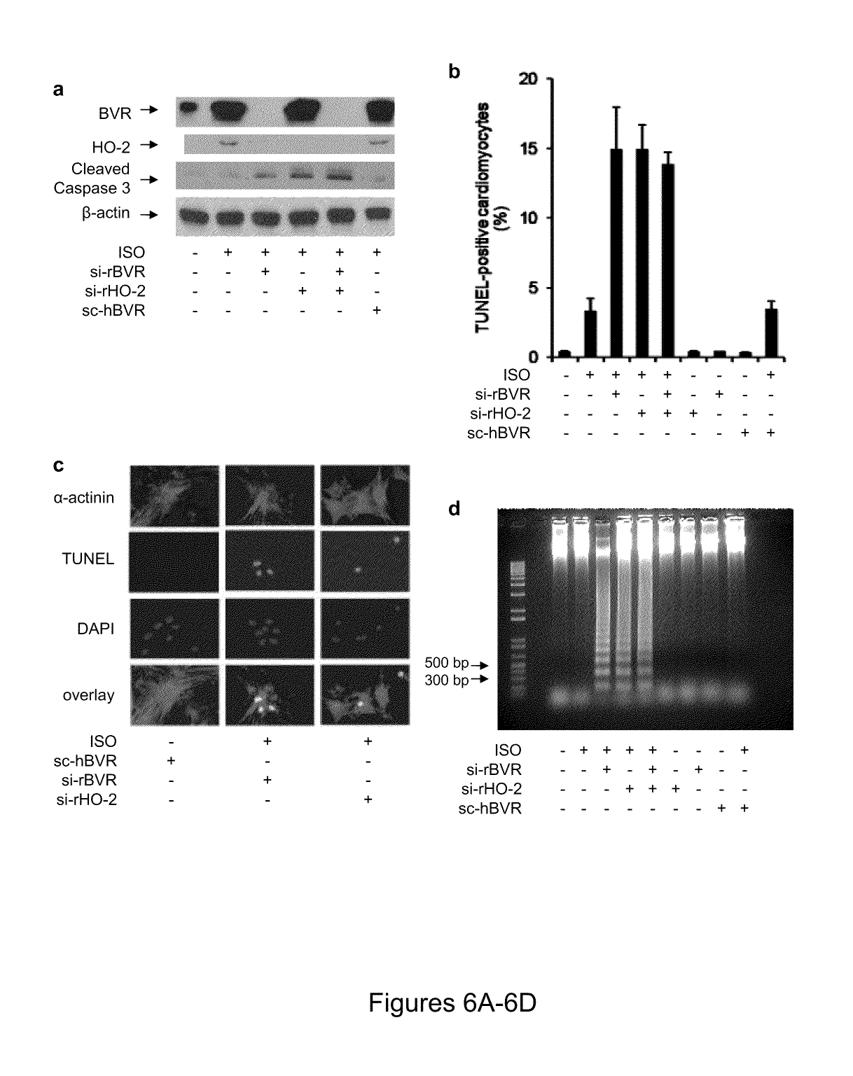

It has been demonstrated that isoproterenol increases cardiomyocyte apoptosis, which thereby mimics the heart in vivo during such pathological conditions as overloading in valvular disease and coronary heart disease (Hu et al., “Chronic Beta-Adrenergic Receptor Stimulation Induces Cardiac Apoptosis and Aggravates Myocardial Ischemia/Reperfusion Injury by Provoking Inducible Nitric-Oxide Synthase-Mediated Nitrative Stress,” J Pharmacol Exp Ther 318:469-475 (2006), which is hereby incorporated by reference in its entirety). It was noted that the level of cleaved caspase-3 protein, a marker for apoptosis, was increased by the inhibition of isoproterenol-induced expression of either BVR or HO-2 using siBVR or si-rHO-2, respectively (FIG. 6A). It was further found that inhibition of protein expression of either BVR or HO-2 by the appropriate siRNA, followed by treatment with isoproterenol, resulted in an increase in the number of cardiac apoptotic cells (FIG. 6B) relative to the extent of apoptosis observed in the absence of ISO stimulation. These data suggest that HO-2 is acting as a heart protective factor during the ISO treatment. That apoptosis was occurring in cardiomyocytes, per se, was determined by triple staining of the cells with anti-α-actinin antibody to detect cardiomyocytes, TUNEL staining for cells undergoing apoptosis, and DAPI to detect nuclei (FIG. 6C). The merged images in FIG. 6C clearly indicate that the cardiomyocytes are undergoing apoptosis. ISO treatment coupled with si-rBVR and si-rHO-2 increased apoptosis to such an extent that DNA fragmentation could be detected by electrophoresis of genomic DNA (FIG. 6D). Simultaneously treating cardiomyocytes with siBVR and siHO-2 did not result in an additive increase in apoptosis compared to treating cardiomyocytes with siBVR or siHO-2 alone. This epistatic effect indicates that the activation of BVR and HO-2 are linked in the same pathway, wherein BVR acts upstream of HO-2, as indicated by the results from the HO-2 stability study (FIG. 5) and the temporal relationship between induction of BVR and HO-2 (FIG. 2). These results suggest that over-expression of HO-2 and BVR could, by reducing apoptosis in cardiomyocytes, be beneficial in preventing heart failure in vivo.

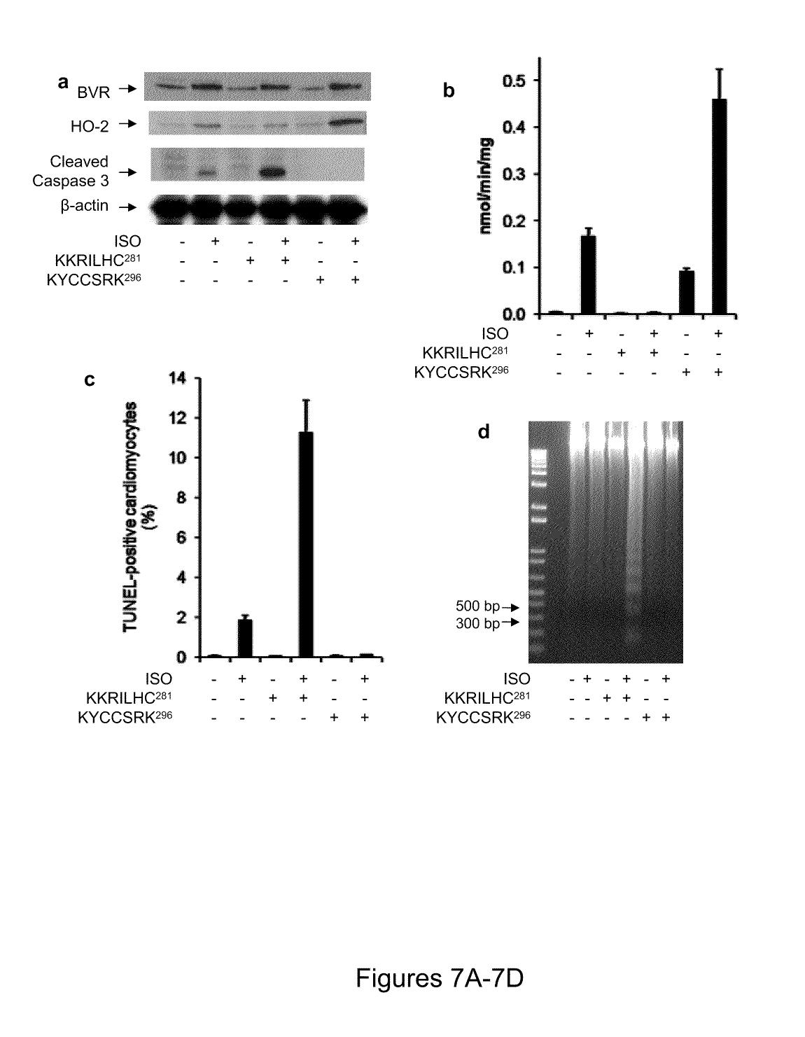

The data thus far suggests that BVR protein expression affects HO-2 protein expression through the regulation of HO-2 stability, which poses the question of whether there is a functional role for enhanced HO-2 expression after BVR activation. Isoproterenol can mimic β-adrenergic activation that occurs as the heart overloads; this phenomenon is universally observed when the heart is under pathophysiological stresses, such as hypertension, valvular diseases and myocardial infarction (Sethi et al., “Inotropic Responses To Isoproterenol in Congestive Heart Failure Subsequent to Myocardial Infarction in Rats,” J Card Fail 1:391-399 (1995); Grimm et al., “Development of Heart Failure Following Isoproterenol Administration in the Rat: Role of the Renin-Angiotensin System,” Cardiovasc Res 37:91-100 (1998); Oudit et al., “Phosphoinositide 3-Kinase Gamma-Deficient Mice Are Protected From Isoproterenol-Induced Heart Failure,” Circulation 108:2147-2152 (2003); and Ferreira et al., “Isoproterenol-Induced Impairment of Heart Function and Remodeling are Attenuated by the Nonpeptide Angiotensin-(1-7) Analogue AVE 0991,” Life Sci 81:916-923 (2007), which are hereby incorporated by reference in their entirety). Previous studies have shown that there are specific sequence elements in hBVR which effectively modulate BVR activities. The peptide KKRILHC²⁸¹ is a potent inhibitor of BVR activity, whereas the peptide KYCCSRK²⁹⁶ is an activator (Lerner-Marmarosh et al., “Regulation of TNF-Alpha-Activated PKC-Zeta Signaling by the Human Biliverdin Reductase: Identification of Activating and Inhibitory Domains of the Reductase,”FASEB J 21:3949-3962 (2007), which is hereby incorporated by reference in its entirety). These peptides may mimic domains in PKC-ζ, resulting in either inhibition or activation, respectively. These peptides were therefore tested using the Langendorff perfusion model to explore the interaction between BVR and HO-2 in the intact heart. Prior to the hearts being prepared for perfusion, the rats received two subcutaneous injections of a low dose of isoproterenol (0.01 mg/kg) separated by a 6 hour interval, to induce BVR activation and protein expression (FIGS. 7A and 7B). The hearts were then isolated and perfused with BVR peptides KKRILHC²⁸¹ or KYCCSRK²⁹⁶, in the presence or absence of isoproterenol.

The peptides KKRILHC²⁸¹ and KYCCSRK²⁹⁶ affect the activity of BVR, but do not affect protein expression, as seen in FIG. 7A. However, as shown in FIG. 7A, peptide KKRILHC²⁸¹ inhibited the BVR-induced HO-2 increase, whereas the peptide KYCCSRK²⁹⁶ increased HO-2 protein expression in ISO-treated heart, above that observed with ISO alone. As shown in FIG. 7B, peptide KKRILHC²⁸¹ blocked the activity of BVR, whether ISO was present or not. Treatment of the heart with peptide KYCCSRK²⁹⁶ alone increased BVR activity; there was, however, a significant activation of BVR reductase activity in hearts treated with both ISO and peptide KYCCSRK²⁹⁶. As shown in FIG. 7C, as measured by the TUNEL assay, ISO increased apoptosis in cardiomyocytes in the intact heart; inhibition of BVR during stimulation by ISO further increased apoptosis to a larger degree. The observed increase in apoptosis was further demonstrated by the elevated levels of cleaved caspase-3 seen in FIG. 7A and the ability to detect nuclear cleavage of genomic DNA by formation of a ladder in FIG. 7D.

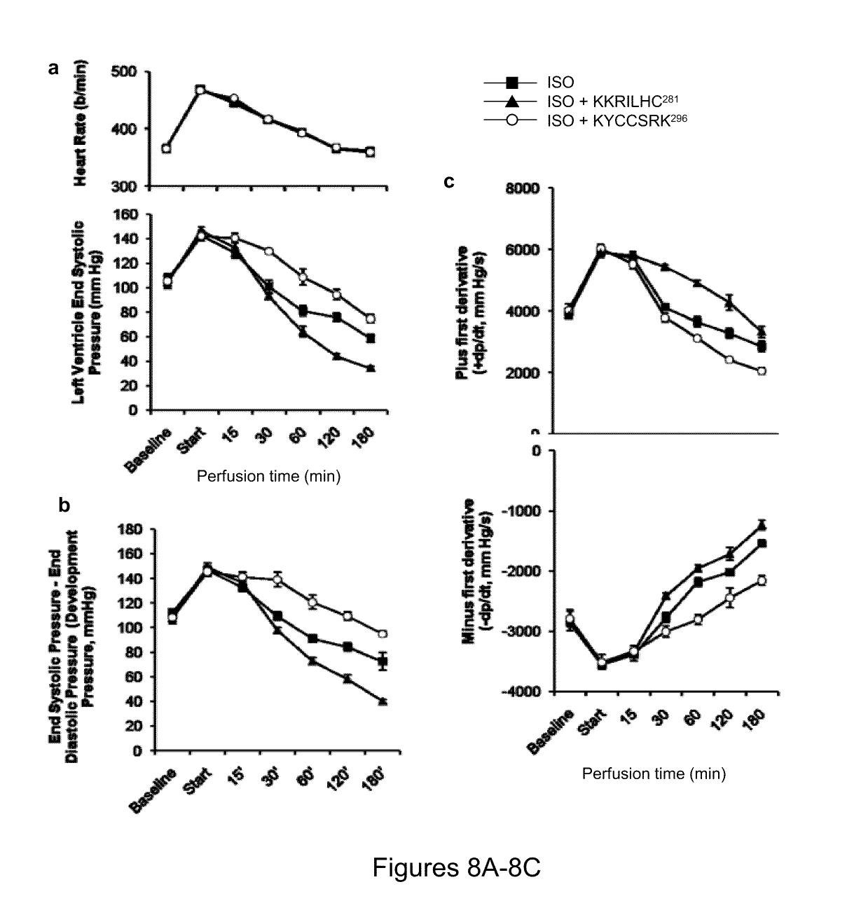

The effect of peptides on the integrity of the heart was also examined in the perfusion system, by measuring left ventricle contractile function in the presence of isoproterenol as a function of time. The heart rate (FIG. 8A, top graph), left ventricular systolic pressure (FIG. 8A, bottom graph, development pressure (FIG. 8B), and the first derivates of systolic pressure (dp/dt, −dp/dt, FIG. 8C) all declined over the course of the experiment. When peptide KKRILHC²⁸¹ was included with isoproterenol in the perfusion buffer, the rate of decline was increased, which could be observed by 30 minutes after initiating perfusion (FIG. 8). This decline in heart function is consistent with the increase in apoptosis (FIG. 7C), observed with this peptide in the presence of ISO. On the other hand, perfusion with the peptide KYCCSRK²⁹⁶ together with isoproterenol slowed the decrease in contractile function observed with isoproterenol alone (FIG. 8). This is consistent with decreased apoptosis and increased BVR activation followed by elevated HO-2 protein expression compared to the isoproterenol control (FIGS. 7, 8). Neither peptide affected the change in heart rate observed with isoproterenol alone (FIG. 8A).

Taken together, this data demonstrates that BVR activity and its subsequent effects on cellular HO-2 protein levels play an important role in the prevention of heart damage that results from β-adrenergic receptor activation.

### Discussion of Examples 1-6

Activation of the β-AR mediates inotropic heart modification and the activation of the apoptotic cell death pathway (Zaugg et al., “Beta-Adrenergic Receptor Subtypes Differentially Affect Apoptosis in Adult Rat Ventricular Myocytes,” Circulation 102:344-350 (2000) and Tomita et al., “Inducible cAMP Early Repressor (ICER) is A Negative-Feedback Regulator of Cardiac Hypertrophy and an Important Mediator of Cardiac Myocyte Apoptosis in Response to Beta-Adrenergic Receptor Stimulation,” Circ Res 93:12-22 (2003), which are hereby incorporated by reference in their entirety). Both β1- and β2-AR subtypes coexist in cardiomyocytes (Bristow et al., “Beta 1- and Beta 2-Adrenergic-Receptor Subpopulations in Nonfailing and Failing Human Ventricular Myocardium: Coupling of Both Receptor Subtypes To Muscle Contraction and Selective Beta 1-Receptor Down-Regulation in Heart Failure,” Circ Res 59:297-309 (1986) and del Monte et al., “Coexistence of Functioning Beta 1- and Beta 2-Adrenoceptors in Single Myocytes From Human Ventricle,” Circulation 88:854-863 (1993), which are hereby incorporated by reference in their entirety); however, the β-AR generated apoptotic signaling is largely dissociated from the β1-AR subtype and selectively mediated by the β2-AR subtype in adult rat ventricular myocytes in vitro (Steinberg S., “The Molecular Basis for Distinct Beta-Adrenergic Receptor Subtype Actions in Cardiomyocytes,” Circ Res 85:1101-1111 (1999) and Nikolaev et al., “Beta2-Adrenergic Receptor Redistribution in Heart Failure Changes cAMP Compartmentation,” Science 327:1653-1657 (2010), which are hereby incorporated by reference in their entirety). Measurement of the response of cardiomyocytes to treatment with ISO cultured cells can be utilized as a model to assess activation of β-ARs upon exposure to pathologic stimuli, such as overloading and ischemia (Sethi et al., “Inotropic Responses to Isoproterenol in Congestive Heart Failure Subsequent to Myocardial Infarction in Rats,” J Card Fail 1:391-399 (1995); Grimm et al., “Development of Heart Failure Following Isoproterenol Administration in the Rat: Role of the Renin-Angiotensin System,” Cardiovasc Res 37:91-100 (1998); Oudit et al., “Phosphoinositide 3-Kinase Gamma-Deficient Mice Are Protected From Isoproterenol-Induced Heart Failure,” Circulation 108:2147-2152 (2003); and Ferreira et al., “Isoproterenol-Induced Impairment of Heart Function and Remodeling are Attenuated by the Nonpeptide Angiotensin-(1-7) Analogue AVE 0991,” Life Sci 81:916-923 (2007), which are hereby incorporated by reference in their entirety).

The above Examples have demonstrated that ISO exposure increases the levels of two enzymes in myocytes, both in vitro and in vivo, which are intimately linked to cellular defense mechanisms—BVR and HO-2. In the intact heart and isolated cardiomyocytes, ISO treatment induces BVR at transcript and protein levels, which in turn leads to an increase in the content of HO-2 protein (FIGS. 2 and 3). BVR induction is a prerequisite for increased myocyte levels of HO-2 (FIG. 5). These data suggest that the increase in HO-2 results from a decrease in the turn-over of HO-2 protein, rather than an increase in HO-2 transcription (FIG. 5). The refractory response of HO-2 transcription to ISO is similar to its response to the host of stimuli that activate HO-1 gene expression. To date only the adrenal glucocorticoids have been shown to induce HO-2 transcription; the only functional regulatory elements identified in the HO-2 promoter are GREs (Raju et al., “Regulation of Heme Oxygenase-2 by Glucocorticoids in Neonatal Rat Brain: Characterization of a Functional Glucocorticoid Response Element,” Biochim Biophys Acta 1351:89-104 (1997), which is hereby incorporated by reference in its entirety. HO-2 is expressed in the endothelial cells of the blood vessels and the sensory cells of the heart (Ewing et al., “Induction of Heart Heme Oxygenase-1 (HSP32) by Hyperthermia: Possible Role in Stress-Mediated Elevation of Cyclic 3′,5′-Guanosine Monophosphate,” J Pharmacol Exp Ther 271:408-414 (1994) and Prabhakar et al., “Carbon Monoxide: A Role in Carotid Body Chemoreception,” Proc Natl Acad Sci USA 92:1994-1997 (1995), which are hereby incorporated by reference in their entirety). Notably, glucocorticoid receptors are expressed in the heart and arterial walls and act directly to maintain vascular tone and response to inflammation and injury (Walker B., “Glucocorticoids and Cardiovascular Disease,” Eur J Endocrinol 157:545-559 (2007), which is hereby incorporated by reference in its entirety).

The HO system, which refers to HO-1, HO-2, and BVR, is a key component of the cellular defense mechanisms against free radical mediated tissue injury, including apoptosis. The system is the sole biological mechanism for the conversion of the pro-oxidant heme to the anti-oxidant bilirubin, the latter being a potent intracellular anti-oxidant (Maghzal et al., “Limited Role for the Bilirubin-Biliverdin Redox Amplification Cycle in the Cellular Antioxidant Protection by Biliverdin Reductase,” J Biol Chem 284:29251-29259 (2009), which is hereby incorporated by reference in its entirety). The second product of heme catabolism, CO, is known for its ability to dilate blood vessels as well to display anti-inflammatory effects (Slebos et al., “Heme Oxygenase-1 and Carbon Monoxide in Pulmonary Medicine,” Respir Res 4:7 (2003); Bolognesi et al., “Carbon Monoxide-Mediated Activation of Large-Conductance Calcium-Activated Potassium Channels Contributes to Mesenteric Vasodilatation in Cirrhotic Rats,” J Pharmacol Exp Ther 321:187-194 (2007); Chung et al., “Interactive Relations Between Nitric Oxide (NO) and Carbon Monoxide (CO): Heme Oxygenase-1/CO Pathway is a Key Modulator in NO-Mediated Antiapoptosis and Anti-Inflammation,” Methods Enzymol 441:329-338 (2008); Ryter et al., “Heme Oxygenase-1/Carbon Monoxide: From Metabolism To Molecular Therapy,” Am J Respir Cell Mol Biol 41:251-260 (2009); and Gozzelino et al., “Mechanisms of Cell Protection by Heme Oxygenase-1,” Annu Rev Pharmacol Toxicol 50:323-354 (2010), which are hereby incorporated by reference in their entirety). While HO-1 and HO-2 both catalyze the formation of CO and biliverdin, which is subsequently converted by BVR to bilirubin, clearly, additional functions of HO-2, its tissue distribution in the heart, as well as its regulatory link to BVR, are likely factors in its selective increase and underscore the significance of its increase. Specifically, HO-2 is a substrate for BVR kinase activity (Lerner-Marmarosh et al., “Human Biliverdin Reductase: A Member of the Insulin Receptor Substrate Family with Serine/Threonine/Tyrosine Kinase Activity,” Proc Natl Acad Sci USA 102:7109-7114 (2005), which is hereby incorporated by reference in its entirety), it functions as an oxygen sensor (Maines M., “The Heme Oxygenase System: A Regulator of Second Messenger Gases,” Annu Rev Pharmacol Toxicol 37:517-554 (1997); Williams et al., “Hemoxygenase-2 is an Oxygen Sensor for a Calcium-Sensitive Potassium Channel,” Science 306:2093-2097 (2004); and Kemp P., “Hemeoxygenase-2 as an O₂Sensor in K⁺ Channel-Dependent Chemotransduction,” Biochem Biophys Res Commun 338:648-652 (2005), which are hereby incorporated by reference in their entirety), and it is expressed in cell types that control response of the organ to oxygen tension (Prabhakar et al., “Carbon Monoxide: A Role in Carotid Body Chemoreception,” Proc Natl Acad Sci USA 92:1994-1997 (1995), which is hereby incorporated by reference in its entirety). HO-2 has the distinctive ability to bind heme to motifs that are not involved in catalytic activity of the oxygenase (McCoubrey et al., “Heme Oxygenase-2 is a Hemoprotein and Binds Heme Through Heme Regulatory Motifs that are not Involved in Heme Catalysis,” J Biol Chem 272:12568-12574 (1997), which is hereby incorporated by reference in its entirety), and hence enables heme binding of gaseous ligands—O₂, CO and NO. Perceivably, sequestration of the pro-inflammatory NO by cellular HO-2 (Ding et al., “Interaction of Heme Oxygenase-2 With Nitric Oxide Donors. Is the Oxygenase an Intracellular ‘Sink’ for NO?” Eur J Biochem 264:854-861 (1999), which is hereby incorporated by reference in its entirety) may be protecting the cells from apoptosis.

Based the current findings, it is apparent that HO-2 plays an important role in protecting cardiomyocytes against apoptosis, which correlates with increase in its cellular content. However, using the currently applied experimental protocols, ISO did not modulate HO-1 expression, therefore, these data do not define the molecular basis for the report by Kinobe et al., “Inhibitors of the Heme Oxygenase—Carbon Monoxide System: On the Doorstep of the Clinic?” Can J Physiol Pharmacol 86:577-599 (2008), which is hereby incorporated by reference in its entirety, which found that HO-1 attenuates ISO-induced cardiac hypertrophy. This, however, is not to question HO-1 mediated attenuation of cardiac hypertrophy induced by angiotensin II (Hu et al., “Heme Oxygenase-1 Inhibits Angiotensin II-Induced Cardiac Hypertrophy In Vitro and In Vivo,” Circulation 110:309-316 (2004), which is hereby incorporated by reference in its entirety) or oxidative stress (Brunt et al., “Heme Oxygenase-1 Inhibits Pro-Oxidant Induced Hypertrophy in HL-1 Cardiomyocytes,” Exp Biol Med (Maywood) 234:582-594 (2009), which is hereby incorporated by reference in its entirety). In addition, induction of HO-1 decreases apoptosis of the cardiomyocytes in ischemia/reperfusion model of heart injury, which has been suggested to reflect inhibition of the transcriptional factors AP-1 and NF-κB (Jadhav et al., “Interaction Among Heme Oxygenase, Nuclear Factor-KappaB, and Transcription Activating Factors in Cardiac Hypertrophy in Hypertension,” Hypertension 52:910-917 (2008) and Yeh et al., “HO-1 Activation Can Attenuate Cardiomyocytic Apoptosis via Inhibition of NF-kappaB and AP-1 Translocation Following Cardiac Global Ischemia and Reperfusion,” J Surg Res 155:147-156 (2009), which are hereby incorporated by reference in their entirety). HO-1 induction may also be a contributing factor to ischemic preconditioning, offering protection against reperfusion injury, perhaps by limiting ROS induction, and thereby preventing against mitochondrial dysfunction (Burwell et al., “Cardioprotection by Metabolic Shut-Down and Gradual Wake-Up,” J Mol Cell Cardiol 46:804-810 (2009), which is hereby incorporated by reference in its entirety).

It has been postulated that there is a necessity for a balance between apoptosis and hypertrophy (van Empel et al., “Myocyte Hypertrophy and Apoptosis: A Balancing Act,” Cardiovasc Res 63:487-499 (2004), which is hereby incorporated by reference in its entirety). While myocardial hypertrophy can normalize wall tension, it also initiates apoptosis and is thus an unfavorable outcome for the heart. Activation of BVR and HO-2 by ISO protects the heart; inhibition of BVR or HO-2 induction with siRNA increases cardiomyocyte apoptosis. However, while ISO increases expression of BVR and HO-2, which serves to limit apoptosis, the induction of these proteins does not eliminate apoptosis entirely. Since apoptosis leads to the loss of the contractile unit in the heart, the cumulative effect of ISO treatment is eventual heart failure. It was previously reported that ISO increases the activity of the apoptotic PDE3-ICER pathway (Ding et al., “A Positive Feedback Loop of Phosphodiesterase 3 (PDE3) and Inducible cAMP Early Repressor (ICER) Leads To Cardiomyocyte Apoptosis,” Proc Natl Acad Sci USA 102:14771-14776 (2005), which is hereby incorporated by reference in its entirety). Thus, it is possible that even though apoptosis is decreased by activation of BVR and HO-2, the alternative PDE3-ICER pathway allows for ongoing cell death, assuming that function of this pathway is largely independent of BVR and HO-2. This does not take into account the possibility that further activation of BVR, above that which is achieved by increased synthesis, might circumvent the alternate pathway and completely protect the cell. The peptides shed light on this possibility Inhibition of BVR activity with the peptide KKRILHC²⁸¹ resulted in a series of detrimental effects on both cardiomyocyte survival and heart function. In these experiments, inhibition of BVR activity virtually eliminated the induction of HO-2, led to significant increases in apoptosis in cultured cells, and resulted in significantly diminished cardiac function in the perfusion assay (FIGS. 7 and 8). On the other hand, activation of BVR could be observed with the peptide KYCCSRK²⁹⁶, and the net effect of this treatment was a greatly enhanced increase of HO-2 protein in the cell, a nearly complete reduction in apoptosis, and improved cardiac function. The effect of the peptide on apoptosis confirms that BVR and/or HO-2 exert their anti-apoptotic effects in cardiomyocytes primarily by interfering with the PDE3-ICER pathway.

Blockade of ISO-mediated induction of BVR by the inhibitor of PKA (PKI) indicates involvement of PKA signaling pathway. At this time, however, the molecular mechanism by which BVR promotes increased cellular content of HO-2 protein is not evident, as it is unclear whether BVR acts directly or by means of an intermediary pathway. Nonetheless, a number of possibilities can be considered. Involvement of the human BVR in regulation of several signaling molecules has been documented. Recent studies have demonstrated that BVR is multifunctional, and can act both as a kinase and as a kinase-kinase (Lerner-Marmarosh et al., “Human Biliverdin Reductase: A Member of the Insulin Receptor Substrate Family with Serine/Threonine/Tyrosine Kinase Activity,” Proc Natl Acad Sci USA 102:7109-7114 (2005); Lerner-Marmarosh et al., “Regulation of TNF-Alpha-Activated PKC-Zeta Signaling by the Human Biliverdin Reductase: Identification of Activating and Inhibitory Domains of the Reductase,” FASEB J 21:3949-3962 (2007); Maines et al., “Human Biliverdin Reductase, a Previously Unknown Activator of Protein Kinase C BetaII,”J Biol Chem 282:8110-8122 (2007); Pachori et al., “Heme-Oxygenase-1-Induced Protection Against Hypoxia/Reoxygenation is Dependent on Biliverdin Reductase and its Interaction With PI3K/Akt Pathway,” J Mol Cell Cardiol 43:580-592 (2007); Florczyk et al., “Biliverdin Reductase: New Features of an Old Enzyme and Its Potential Therapeutic Significance,” Pharmacol Rep 60:38-48 (2008); Lerner-Marmarosh et al., “Human Biliverdin Reductase is an ERK Activator; hBVR is an ERK Nuclear Transporter and is Required for MAPK Signaling,” Proc Natl Acad Sci USA 105:6870-6875 (2008); and Wegiel et al., “Cell Surface Biliverdin Reductase Mediates Biliverdin-Induced Anti-Inflammatory Effects Via Phosphatidylinositol 3-Kinase and Akt,” J Biol Chem 284:21369-21378 (2009), which are hereby incorporated by reference in their entirety. For example, BVR has been shown to activate PKC-βII—indeed, there is a distinct possibility that BVR is the PKC-βII-kinase (Maines et al., “Human Biliverdin Reductase, a Previously Unknown Activator of Protein Kinase C BetaII,”J Biol Chem 282:8110-8122 (2007), which is hereby incorporated by reference in its entirety). BVR also functions in the TNF-α-activated PKC-ζ signaling pathway by binding to and, thus, activating the PKC (Lerner-Marmarosh et al., “Regulation of TNF-Alpha-Activated PKC-Zeta Signaling by the Human Biliverdin Reductase: Identification of Activating and Inhibitory Domains of the Reductase,” FASEB J 21:3949-3962 (2007), which is hereby incorporated by reference in its entirety). BVR can function as an intermediary to link the MAPK and PI3K pathways (Maines et al., “Human Biliverdin Reductase, a Previously Unknown Activator of Protein Kinase C BetaII,”J Biol Chem 282:8110-8122 (2007), and Lerner-Marmarosh et al., “Human Biliverdin Reductase is an ERK Activator; hBVR is an ERK Nuclear Transporter and is Required for MAPK Signaling,” Proc Natl Acad Sci USA 105:6870-6875 (2008), which are hereby incorporated by reference in their entirety). In addition, using purified preparations of HO-2 and BVR (Lerner-Marmarosh et al., “Human Biliverdin Reductase: A Member of the Insulin Receptor Substrate Family with Serine/Threonine/Tyrosine Kinase Activity,” Proc Natl Acad Sci USA 102:7109-7114 (2005), which is hereby incorporated by reference in its entirety), it has been demonstrated that the reductase functions as a kinase for the oxygenase. Accordingly, any of the noted signaling pathways may be involved in HO-2 increase.

Should increases in HO-2 be a component of the package of benefits offered by the glucocorticoids to the cardiovascular system, then the findings that (i) activation of BVR leads to an increase in HO-2 levels in the heart, and (ii) administration of a seven amino acid human BVR-based peptide results in a near complete reduction in apoptosis and improved cardiac function (FIGS. 7 and 8) provide a novel approach for the treatment of cardiovascular disease.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a coronary disorder comprising:
administering to a patient having a coronary disorder an effective amount of an agent that induces the stabilization of heme oxygenase-2 (HO-2) in cardiac cells, whereby said administering is effective to increase the level of HO-2 in cardiac cells and thereby treat the coronary disorder.

2. The method according to claim 1, wherein the coronary disorder is myocardial infarction or stroke.

3. The method according to claim 1, wherein the coronary disorder is chronic coronary ischemia, arteriosclerosis, congestive heart failure, angina, atherosclerosis, myocardial hypertrophy, dilated cardiomyopathy, restenosis, coronary artery disease, arrhythmia, angina, or hypertension.

4. The method according to claim 1, wherein the patient is a mammal.

5. The method according to claim 1, wherein the agent is a mammalian biliverdin reductase (“BVR”) or a BVR peptide fragment thereof.

6. The method according to claim 5, wherein the BVR peptide fragment comprises an amino acid sequence of KX[C/H][C/H]SXK (SEQ ID NO:7), wherein X at position 2 is Y, T, or S and X at position 6 is a positively charged amino acid, preferably, R or K.

7. The method according to claim 6, wherein the BVR peptide is selected from the group consisting of KYCCSRK (SEQ ID NO:8), KSCCSRK (SEQ ID NO:9), KTCCSRK (SEQ ID NO:10), KYCCSKK (SEQ ID NO:11), KSCCSKK (SEQ ID NO:12), KTCCSKK (SEQ ID NO:13), KYHCSRK (SEQ ID NO:14), KSHCSRK (SEQ ID NO:15), KTHCSRK (SEQ ID NO:16), KYHCSKK (SEQ ID NO:17), KSHCSKK (SEQ ID NO:18), and KTHCSKK (SEQ ID NO:19).

8. The method according to claim 6, wherein the BVR peptide is myristolated or acetylated.

9. The method according to claim 1, wherein the agent is an expression system that encodes a mammalian biliverdin reductase (“BVR”) or a BVR peptide fragment.

10. The method according to claim 9, wherein the expression system comprises a cardiac cell-specific promoter or an inducible promoter.

11. A method of inducing cancer cell death in a patient comprising:
administering to a patient having cancer, an effective amount of an agent that induces the destabilization of heme oxygenase-2 (HO-2) in cancer cells, whereby said administering is effective to decrease the level of HO-2 in the cancer cells of the patient and thereby induce cancer cell death in the patient.

12. The method according to claim 11, wherein the cancer cells are selected from the group consisting of ovarian cancer cells, esophageal cancer cells, colorectal cancer cells, prostate cancer cells, renal cancer cells, pancreatic cancer cells, breast cancer cells, skin cancer cells, oral cavity cancer cells, lung cancer cells, gastrointestinal cancer cells, liver cancer cells, head and neck cancer cells, and brain cancer cells.

13. The method according to claim 11, wherein the patient is a mammal.

14. The method according to claim 11, wherein the agent inhibits biliverdin reductase.

15. The method according to claim 14, wherein the agent is an inhibitory BVR nucleic acid selected from the group consisting of siRNA, shRNA, and antisense RNA.

16. The method according to claim 14, wherein the agent is an inhibitory BVR peptide.

17. The method according to claim 16, wherein the inhibitory BVR peptide comprises an amino acid sequence of XXX[I/L]LXX (SEQ ID NO:20), wherein X at positions 1, 2, and 3 is a positively charged amino acid residue, preferably K or R, X at position 6 is any amino acid, preferably C or H, and X at position 7 is any amino acid, preferably C.

18. The method according to claim 17, wherein the inhibitory BVR peptide is selected from the group consisting of KKRILHC (SEQ ID NO:21), RKRILCC (SEQ ID NO:22), KRRILCC (SEQ ID NO:23), KKRLLCC (SEQ ID NO:24), RRRILCC (SEQ ID NO:25), KRKILCC (SEQ ID NO:26), RRRLLCC (SEQ ID NO:27), and KKKLLHC (SEQ ID NO:28).

19. The method according to claim 11 wherein the agent comprises an expression system that encodes an inhibitory BVR peptide.

20. An isolated peptide comprising the amino acid sequence KX[C/H][C/H]SXK (SEQ ID NO:7), wherein X at position 2 is Y, T, or S and X at position 6 is a positively charged amino acid, preferably, R or K.

21. The isolated peptide according to claim 20, wherein the peptide consists of SEQ ID NO:7.

22. The isolated peptide according to claim 20, wherein the peptide is acetylated or myristoylated.

23. The isolated peptide according to claim 20, wherein the peptide is selected from the group consisting of KYCCSRK (SEQ ID NO:8), KSCCSRK (SEQ ID NO:9), KTCCSRK (SEQ ID NO:10), KYCCSKK (SEQ ID NO:11), KSCCSKK (SEQ ID NO:12), KTCCSKK (SEQ ID NO:13), KYHCSRK (SEQ ID NO:14), KSHCSRK (SEQ ID NO:15), KTHCSRK (SEQ ID NO:16), KYHCSKK (SEQ ID NO:17), KSHCSKK (SEQ ID NO:18), and KTHCSKK (SEQ ID NO:19).

24. A pharmaceutical composition comprising the isolated peptide according to claim 20 and a pharmaceutically acceptable carrier.

25. An isolated peptide comprising the amino acid sequence) XXX[I/L]LXX (SEQ ID NO:20), wherein X at positions 1, 2, and 3 is a positively charged amino acid residue, preferably K or R, X at position 6 is any amino acid, preferably C or H, and X at position 7 is any amino acid, preferably C.

26. The isolated peptide according to claim 25, wherein the peptide consists of SEQ ID NO:20.

27. The isolated peptide according to claim 25, wherein the peptide is acetylated or myristoylated.

28. The isolated peptide according to claim 25, wherein the peptide is selected from the group consisting of KKRILHC (SEQ ID NO:21), RKRILCC (SEQ ID NO:22), KRRILCC (SEQ ID NO:23), KKRLLCC (SEQ ID NO:24), RRRILCC (SEQ ID NO:25), KRKILCC (SEQ ID NO:26), RRRLLCC (SEQ ID NO:27), and KKKLLHC (SEQ ID NO:28).

29. A pharmaceutical composition comprising the isolated peptide according to claim 25 and a pharmaceutically acceptable carrier.
